(19) United States
(12) Patent Application Publication
(10) Pub. No.: US 2014/0127672 A1
(43) Pub. Date: May 8, 2014

(54) DISEASE DETECTION IN PLANTS

(71) Applicants: Cristina Elizabeth Davis, Davis, CA (US); Oliver Fiehn, Davis, CA (US); Abhaya M. Dandekar, Davis, CA (US); Alexander Aksenov, Davis, CA (US); Weixiang Zhao, Davis, CA (US); William Cheung, Davis, VA (US); Federico Martinelli, Davis, CA (US); Kirsten Jean Skogerson, Davis, CA (US)

(72) Inventors: Cristina Elizabeth Davis, Davis, CA (US); Oliver Fiehn, Davis, CA (US); Abhaya M. Dandekar, Davis, CA (US); Alexander Aksenov, Davis, CA (US); Weixiang Zhao, Davis, CA (US); William Cheung, Davis, VA (US); Federico Martinelli, Davis, CA (US); Kirsten Jean Skogerson, Davis, CA (US)

(73) Assignee: The Regents of the University of California, Oakland, CA (US)

(21) Appl. No.: 14/006,653

(22) PCT Filed: Mar. 21, 2012
(86) PCT No.: PCT/US12/30003

§ 371 Date: Dec. 2, 2013

Related U.S. Application Data
(60) Provisional application No. 61/465,649, filed on Mar. 21, 2011.

(51) Int. Cl. G01N 33/487; G01N 27/62; C12Q 1/68

(52) U.S. Cl. CPC G01N 33/48707; C12Q 1/689; G01N 27/624
USPC 435/5; 435/34; 435/6.12

(57) ABSTRACT

The present disclosure relates to disease detection in plants. In particular, it provides methods, compositions, and devices for the detection of diseases in plants.

### CROSS-REFERENCE TO RELATED APPLICATION

This application claims the benefit of U.S. Provisional Application No. 61/465,649, filed Mar. 21, 2011, which is hereby incorporated by reference in its entirety.

### FIELD

The present disclosure relates generally to disease detection in plants, and, more particularly, to methods and systems for the early detection of infectious diseases in citrus plants.

### SUBMISSION OF TABLES ON ASCII TEXT FILE

The content of the following submissions on ASCII text file are incorporated herein by reference in their entirety: computer readable forms (CRF) of Tables 1-15 (file names: Table1.txt, containing 134 KB; Table2.txt, containing 93 KB; Table3.txt, containing 94 KB; Table4.txt, containing 25 KB; Table5.txt, containing 48 KB; Table6.txt, containing 65 KB; Table7.txt, containing 21 KB; Table8.txt, containing 55 KB; Table9.txt, containing 140 KB; Table10.txt, containing 119 KB; Table11.txt, containing 85 KB; Table12.txt, containing 113 KB; Table13.txt, containing 94 KB; Table14.txt, containing 104 KB; and Table15.txt, containing 6 KB). These files were created on Mar. 21, 2011.

### BACKGROUND

Plant pathogens pose significant challenges to the agricultural industry in many countries by devastating crops worldwide.

An example of disease which has a major economic impact is “citrus greening” disease, or Huanglongbing (HLB), which has been devastating to citrus crops in South East Asia, Brazil and USA. HLB, a vector-borne plant disease, is caused by the bacterium Candidatus Liberibacter asiaticus (CaLas) and spread by phloem-feeding insect Asian citrus psyllid. Although not harmful to human health, HLB is devastating to citrus plants due to its effect on production, tree decline, and fruit size and shape. Sweet oranges, mandarins, and tangelos are highly susceptible, followed by sour oranges, grapefruits, and other commercially important citrus varieties. Only a few lemon cultivars and a few other species like Citrus indica and Citrus macroptera reportedly displayed some tolerance or possibly resistance to the bacterium.

Candidatus Liberibacter is a member of the alpha subdivision of the proteobacteria, based on ribosomal region sequence data (Jagoueix et al., 1994). CaLas, transmitted by the Asian citrus psyllid Diaphorina citri, lives in the phloem of infected citrus and, once acquired, is transmitted for the life of the insect vector. Insecticides can reduce psyllid populations, but because the bacterium persists in the vector, a few psyllids alone can spread the disease.

Since citrus plants remain asymptomatic for the disease over long periods, it is important to identify the infection before symptoms appear. If detected at an early stage, transmission of the disease from infected trees can be halted or diminished via selective tree removal in commercial orchards. Infected trees can be provided with elevated nutrient therapy to minimize symptom and decline development, as well.

To date, there are few confirmatory methods to detect citrus infection with the Candidatus Liberibacter bacterium if it is asymptomatic. Polymerase chain reaction (PCR) testing is one potential method for diagnosing HLB. However, PCR is an expensive and time-consuming process that is further challenging because the bacterial loads in plants are distributed unevenly and can fluctuate with time.

Since CaLas lives in phloem, this route is probably responsible for rapid bacterial spread after a portion of the tree is infected. For this reason, it has been hypothesized that CaLas is present in asymptomatic leaves of infected trees, but at concentrations near or below the PCR detection limit (4.6×102 l/g).

Another plant pathogen that causes a major economic impact is Citrus tristeza virus (CTV). CTV is a plant based virus belonging to the genus Closterovirus, family Closteroviridae, and it is adapted to replicate within the phloem tissue of its host. CTV has a filamentous structure of approximately 2000 nm in length and 10-12 nm in diameter. Its RNA genome is estimated to be about 20 Kb in size and it was first sequenced in 1995 by Karsaev et al (Kersev 1995). CTV is considered to have one of the largest genome of any known plant virus. CTV predominately infects plants within the Rutacese family, which includes economically important fruit crops such as sweet oranges, Clementines, limes and grapefruits cultivars. These cultivar species are propagated by grafting new rootstock onto existing scion. Thus, any infected budwood and rootstock acts as an artificial vector, introducing the virus to new regions, which are then spread on a local level by aphids, whiteflies and mealybug. Within the last 70 years it has been estimated that over 80 million trees, primary Clementine and sweet orange varieties have been destroyed due CTV infection worldwide. To date the CTV represent a real and significant economic burden to the citrus industry.

CTV infected crops develop three noticeable symptoms, depending on the host species infected and the scion-rootstock combinations: (1) Seeding yellow (SY) is characterized by chlorotic leaves, reduction of the infected host's root system and production of lower quality fruit; (2) Quick decline (QD) is induced by necrosis at the inference between the scion/rootstock, causing initially wilted leaves and reduced foliage, followed by eventual death of the entire citrus tree within weeks after initial symptoms appear. (3) Stem pitting (SP) is rarely associated to be fatal to host plants but significantly reduces vigor which, in turn, drastically affects crop yield production: those symptoms are noticable even in disease tolerant rootstock.

Measures to control CTV infection of citrus crops includes quarantine, establishment of budwood certification programs, removal and elimination of infected trees and the incorporation of tolerant rootstock. This is dependent upon the severity and size of the infected areas/regions.

To date, the CTV is extensively characterized both biologically and at the molecular level (Bruessow 2010). Methods employed to detect CTV infections in citrus fruit crops include viral indexing by testing with certain lime cultivars, electron microscopy (EM) (Bar-Joseph 1979), real time reverse transcriptase polymerase chain reaction (RT-PCR), and spectroscopic analysis (FT-IR)

Provided herein are improved methods and compositions for the detection of disease in plants.

### SUMMARY

The present disclosure relates to detecting plant diseases such as Huanglongbing (HLB) and Citrus tristeza virus (CTV) in plants by analysis of plant volatile compounds VOCs. The present disclosure also relates to detecting plant diseases such as Huanglongbing (HLB) and Citrus tristeza virus (CTV) in plants by analysis of plant gene expression.

In one embodiment, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample; and c) comparing the measured quantity of the one or more volatile chemical compounds to a predetermined value of the one or more volatile chemical compounds to diagnose Huanglongbing disease in the citrus plant.

In one embodiment, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein the predetermined value is determined by measuring the quantity of the one or more volatile chemical compounds released by a reference citrus plant infected with Huanglongbing disease.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein the predetermined value is determined by measuring the quantity of the one or more volatile chemical compounds released by a reference citrus plant not infected with Huanglongbing disease.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein the predetermined value is determined by measuring the quantity of the one or more volatile chemical compounds released by a reference citrus plant infected with Huanglongbing disease, wherein the reference citrus plant is at the same developmental stage as the citrus plant.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein the predetermined value is determined by measuring the quantity of the one or more volatile chemical compounds released by a reference citrus plant not infected with Huanglongbing disease, wherein the reference citrus plant is at the same developmental stage as the citrus plant.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein a mass spectrometer is used to measure the quantity of one or more volatile chemical compounds in the sample.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein a differential mobility spectrometer is used to measure the quantity of one or more volatile chemical compounds in the sample.

In one aspect, provided herein is a method of diagnosing Huanglongbing disease in a citrus plant, the method including: a) obtaining a sample of volatile chemical compounds released by the citrus plant; b) measuring the quantity of one or more volatile chemical compounds, in the sample, selected from the group of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and c) comparing the measured quantity of the one or more volatile chemical compounds to a predetermined value of the one or more volatile chemical compounds to diagnose Huanglongbing disease in the citrus plant, wherein the citrus plant is a Valencia orange plant.

In one embodiment, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample; and c) comparing the measured quantity of the one or more volatile chemical compounds to a predetermined value of the one or more volatile chemical compounds to diagnose CTV disease in the citrus plant.

In one embodiment, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample, selected from the group of: myrcene, carene (delta-3-), ocimene (e-beta-), hexadecanol, limonene, tetracosane, bulnesene (alpha-), and c) comparing the measured quantity of the one or more volatile chemical compounds to a predetermined value of the one or more volatile chemical compounds to diagnose CTV disease in the citrus plant.

In one aspect, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample, selected from the group of: myrcene, carene (delta-3-), ocimene (e-beta-), hexadecanol, limonene, tetracosane, bulnesene (alpha-), and c) comparing the measured quantity of the one or more volatile chemical compounds to a predetermined value of the one or more volatile chemical compounds to diagnose CTV disease in the citrus plant, wherein the predetermined value is determined by measuring the quantity of the one or more volatile chemical compounds released by a reference citrus plant infected with CTV.

In one aspect, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample, selected from the group of: myrcene, carene (delta-3-), ocimene (e-beta-), hexadecanol, limonene, tetracosane, bulnesene (alpha-), and c) comparing the measured quantity of the one or more volatile chemical compounds to a predetermined value of the one or more volatile chemical compounds to diagnose CTV disease in the citrus plant, wherein the predetermined value is determined by measuring the quantity of the one or more volatile chemical compounds released by a reference citrus plant not infected with CTV.

In one aspect, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample, selected from the group of: myrcene, carene (delta-3-), ocimene (e-beta-), hexadecanol, limonene, tetracosane, bulnesene (alpha-), and c) comparing the measured quantity of the one or more volatile chemical compounds to a predetermined value of the one or more volatile chemical compounds to diagnose CTV disease in the citrus plant, wherein the predetermined value is determined by measuring the quantity of the one or more volatile chemical compounds released by a reference citrus plant infected with CTV, and wherein the reference citrus plant is at the same developmental stage as the citrus plant.

In one aspect, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample, selected from the group of: myrcene, carene (delta-3-), ocimene (e-beta-), hexadecanol, limonene, tetracosane, bulnesene (alpha-), and c) comparing the measured quantity of the one or more volatile chemical compounds to a predetermined value of the one or more volatile chemical compounds to diagnose CTV disease in the citrus plant, wherein the predetermined value is determined by measuring the quantity of the one or more volatile chemical compounds released by a reference citrus plant not infected with CTV, and wherein the reference citrus plant is at the same developmental stage as the citrus plant.

In one aspect, provided herein is a method of diagnosing Citrus tristeza virus (CTV) in a citrus plant, the method including: a) obtaining a sample of volatile chemical compounds released by the citrus plant; b) measuring the quantity of one or more volatile chemical compounds, in the sample, selected from the group of: myrcene, carene (delta-3-), ocimene (e-beta-), hexadecanol, limonene, tetracosane, bulnesene (alpha-), and c) comparing the measured quantity of the one or more volatile chemical compounds to a predetermined value of the one or more volatile chemical compounds to diagnose CTV disease in the citrus plant, wherein a mass spectrometer and/or a differential mobility spectrometer is used to measure the quantity of one or more volatile chemical compounds in the sample.

In one embodiment, provided herein is a method of diagnosing Huanglongbing disease in a citrus plant, the method including: a) obtaining a sample of nucleic acid molecules generated by the citrus plant; b) measuring the quantity of one or more nucleic acid molecules in the sample; and c) comparing the measured quantity of the one or more nucleic acid molecules to a predetermined value of the one or more nucleic acid molecules to diagnose Huanglongbing disease in the citrus plant.

In one embodiment, provided herein is a method of diagnosing Huanglongbing disease in a citrus plant, the method including: a) obtaining a sample of nucleic acid molecules generated by the citrus plant; b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (844256583); U-box (822566824); HSP82 (844237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and c) comparing the measured quantity of the one or more nucleic acid molecules to a predetermined value of the one or more nucleic acid molecules to diagnose Huanglongbing disease in the citrus plant.

In one aspect, provided herein is a method of diagnosing Huanglongbing disease in a citrus plant, the method including: a) obtaining a sample of nucleic acid molecules generated by the citrus plant; b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (S44256583); U-box (S22566824); HSP82 (S44237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and c) comparing the measured quantity of the one or more nucleic acid molecules to a predetermined value of the one or more nucleic acid molecules to diagnose Huanglongbing disease in the citrus plant, wherein the nucleic acid molecules are mRNA molecules.

In one aspect, provided herein is a method of diagnosing Huanglongbing disease in a citrus plant, the method including: a) obtaining a sample of nucleic acid molecules generated by the citrus plant; b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (S44256583); U-box (S22566824); HSP82 (S44237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and c) comparing the measured quantity of the one or more nucleic acid molecules to a predetermined value of the one or more nucleic acid molecules to diagnose Huanglongbing disease in the citrus plant, wherein the predetermined value is determined by measuring the quantity of the one or more nucleic acid molecules generated by a reference citrus plant infected with Huanglongbing disease.

In one aspect, provided herein is a method of diagnosing Huanglongbing disease in a citrus plant, the method including: a) obtaining a sample of nucleic acid molecules generated by the citrus plant; b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (S44256583); U-box (S22566824); HSP82 (S44237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and c) comparing the measured quantity of the one or more nucleic acid molecules to a predetermined value of the one or more nucleic acid molecules to diagnose Huanglongbing disease in the citrus plant, wherein the predetermined value is determined by measuring the quantity of the one or more nucleic acid molecules generated by a reference citrus plant not infected with Huanglongbing disease.

In one aspect, provided herein is a method of diagnosing Huanglongbing disease in a citrus plant, the method including: a) obtaining a sample of nucleic acid molecules generated by the citrus plant; b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (S44256583); U-box (S22566824); HSP82 (S44237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and c) comparing the measured quantity of the one or more nucleic acid molecules to a predetermined value of the one or more nucleic acid molecules to diagnose Huanglongbing disease in the citrus plant, wherein the predetermined value is determined by measuring the quantity of the one or more nucleic acid molecules generated by a reference citrus plant infected with Huanglongbing disease, wherein the reference citrus plant is at the same developmental stage as the citrus plant.

In one aspect, provided herein is a method of diagnosing Huanglongbing disease in a citrus plant, the method including: a) obtaining a sample of nucleic acid molecules generated by the citrus plant; b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (S44256583); U-box (S22566824); HSP82 (S44237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and c) comparing the measured quantity of the one or more nucleic acid molecules to a predetermined value of the one or more nucleic acid molecules to diagnose Huanglongbing disease in the citrus plant, wherein the predetermined value is determined by measuring the quantity of the one or more nucleic acid molecules generated by a reference citrus plant not infected with Huanglongbing disease, wherein the reference citrus plant is at the same developmental stage as the citrus plant.

### BRIEF DESCRIPTION OF THE DRAWINGS

FIG. 1—GC/DMS plot showing relative ion counts of VOCs from HLB-infected Hamlin orange tree plotted against GC retention time and compensation voltage (CV).

FIG. 2—Wavelet transformation use to extract information from GC/DMS data. Raw spectral data can be decomposed into low frequency parts and high frequency parts.

FIG. 3—Fragment of GC profiles for healthy (bottom line) vs. HLB-infected symptomatic (top line) Hamlin orange trees.

FIG. 4—Identification of volatile metabolites in a volatile BinBase database (“vocBB”) by using retention index and mass spectral matching with the Adams library. Panel (A) shows correlation of Adams index with Fiehn index; Panel (B) shows MS spectrum in the database corresponding to methyl salicylate.

FIG. 5—Cluster diagram of four citrus essential oils used to populate the volatile BinBase database. All essential oils created novel entries in the database. All samples were correctly clustered by the vocBB data processing.

FIG. 6—Volatile profiling in HLB infected plants, Ft. Pierce, Fla. Left panel: 6 weeks after infection, bagged leaves with Twister volatile adsorption. Right panel: Monitoring of disease progression by PCR and symptom description.

FIG. 7—Temporal emission of citrus volatiles during plant development, both for healthy and HLB infected plants.

FIG. 8—Number of significantly different metabolites (p<0.05) between healthy and HLB infected citrus plants. Square-marked line: all compounds; diamond-marked line: compounds with identified structures.

FIG. 9—Examples of significantly different metabolites (p<0.05) (indicated by stars) between healthy and HLB infected citrus plants. Top: identified volatiles; bottom: structurally unidentified volatiles.

FIG. 10—HLB-regulation of pathways associated with scent and aroma compounds.

FIG. 11—HLB-regulation of specific genes involved in Jasmonate biosynthesis and validation using qRT-PCR. CO=healthy; AH=apparently healthy; AS=asymptomatic; and SY=symptomatic plants.

FIG. 12—Validation of terpenoid pathways in mature fruit: qRT-PCR analysis of two genes in this pathway. CO=healthy; AH=apparently healthy; AS=asymptomatic; and SY=symptomatic plants.

FIG. 13—Validation of terpenoid pathways in young and mature leaves: qRT-PCR analysis of two genes in this pathway. CO=healthy; AH=apparently healthy; AS=asymptomatic; and SY=symptomatic plants.

FIG. 14—Identification of monoterpene compounds as potential biomarkers for early detection of HLB disease.

FIG. 15—qRT-PCR validation of up-regulation of salicylic acid methyl transferase, an early biomarker for HLB disease in leaves and fruit.

FIG. 16—Network of genes/pathways regulated by HLB in fruit.

FIG. 17—Part (A) HLB-regulated genes involved in ubiquitin-dependent degradation processes; part (B) qRT-PCR analysis of HSP82 in fruit; part (C) protein-protein interaction network deduced in citrus from Arabidopsis knowledgebase.

FIG. 18—Principal component analysis (“PCA”) score plot of healthy plants vs. plants infected with CTV only (“Healthy vs. CTV only”): Separation between the healthy and CTV infected can be observed, and with the exception of some possible outliers, the two classes can be broadly separated.

FIG. 19—PCA score plot of all three classes: Healthy, CTV, and CTV+Stubborn. A clear separation can still be observed between Healthy and all the CTV relevant samples, although there is a significant overlap between the CTV and both CTV+stubborn.

FIG. 20—Three examples of chemicals that have a strong relationship with CTV: myrcene, carene (delta-3-), and ocimene (e-beta-). These chemicals are 3 out of 18 chemicals that are present in both the “Healthy vs. CTV only” (FIG. 18) and the “Healthy vs. CTV relevant” (FIG. 19).

FIG. 21—HLB detection based on SPME GC/MS HLB biomarkers (Florida). Separation based on the nine distinguishable biomarkers can be observed between HLB-diseased and healthy specimens.

FIG. 22—VOCs from selected plant leaves are monitored using two parallel chemical analysis systems, the gas chromatograph mass spectrometer (GC/ITMS) and differential mobility spectrometer (GC/DMS). The retention times from the spectral outputs are paired and indexed, and equivalent spectra features in both of the data sets can be located to identify putative biomarkers of citrus health and disease.

FIG. 23—GC/ITMS (TIC) spectra of the two related varietals Washington Navel and Valencia, offset to the same scale.

FIG. 24—Three mass spectra from each varietal (Washington Navel and Valencia) show against each other, expanded view of the prominent peaks present in both samples. The top 3 spectra are from Washington Navel, the bottom 3 spectra are from Valencia.

FIG. 25—Box and whisker plots of the log-normalized concentrations of VOC markers and their distribution profiles across the two varietals Washington Navel and Valencia: Peak 13 (RT 68.464 min), peak 18 (RT 75.037 min), peak 24 (RT 121.567 min) and peak 34 (RT 141.903)

FIG. 26—PCA score plot based on the GC/ITMS data, using the four variable identified to be significant through a Student t-test comparison: the total explained variance captured by each principal component is shown.

FIG. 27—Total ion chromatogram and GC/DMS spectra from analysis of a Valencia leaf headspace.

FIG. 28—The mean signal intensities of Valencia and W. Navel and their difference per GC/DMS spectra (top panels: positive ion spectra; bottom panels: negative ion spectra).

FIG. 29—Distribution of the principal components based on the GC/DMS data and Student's t-test selected pixels based on the (A) positive ion spectra only, (B) negative ion spectra only, and (C) both ion spectra simultaneously: (+: Valencia, o: Washington Navel). The variances explained by principal components 1 and 2 is (A: 49% and 14.0%), (B: 40.0% and 17.0%), and (C: 43.6% and 16.0%).

FIG. 30—Sum dot product values for all the SPME-GC/ITMS spectra for Washington Naval varietals (TIC).

FIG. 31—Sum dot product values for all the SPME-GC/ITMS spectra for Valencia varietals.

FIG. 32—Using a suitcase-sized portable GC/DMS sensor for field sampling. Left panel: a brief display of the portable GC/DMS structure; Middle panel: field sampling and analysis with the portable GC/DMS sensor; Right panel: solar panel for power supply.

FIG. 33—Benchmark study of plant category separation using the portable GC/DMS. Panel A: a GC/DMS plot of the VOC from plant leaf; Panel B: principal component analysis of the plant category separation.

FIG. 34—P-value of Student's t-test (p<0.1) across the whole signal domain

FIG. 35—Loading coefficients of each pixel for the top 3 principal components

FIG. 36—Average spectra for Healthy and HLB.

FIG. 37—Separation between healthy and CTV

FIG. 38 Separation between HLB and healthy samples based on the wavelet coefficients of GC/DMS signal of the whole retention time range (left panel) and the first three minutes (right panel).

FIG. 39—Separation between healthy and CTV samples using the wavelet coefficients of their GC/DMS signal.

FIG. 40—Chromatograms showing relative total ions counts of VOCs from healthy and disease Valencia and Hamlin orange trees.

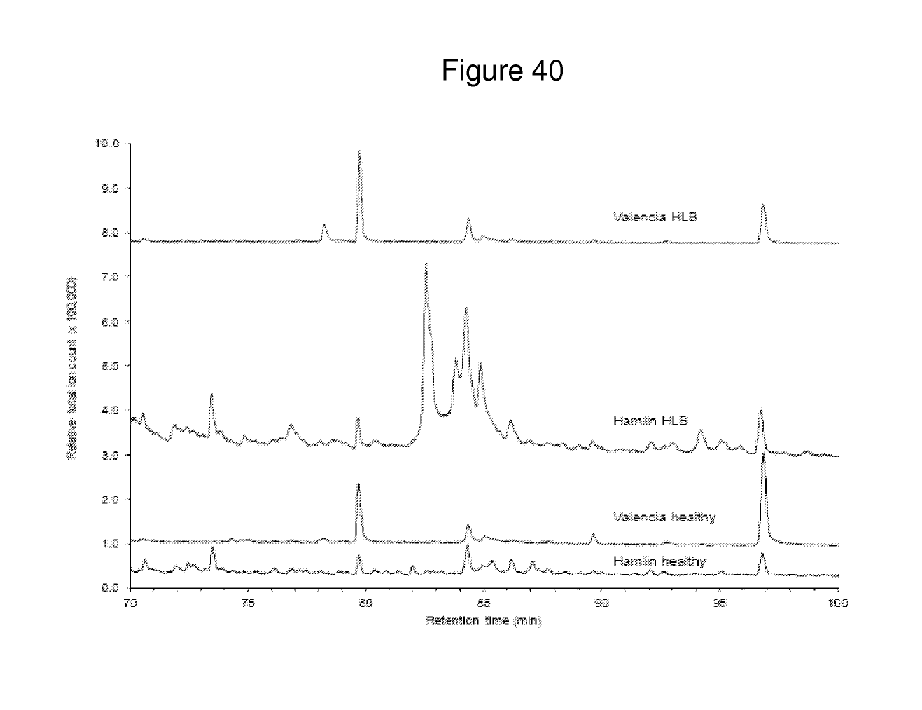

### DETAILED DESCRIPTION OF EMBODIMENTS

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The present disclosure relates to the diagnosis of disease in plants.

### Diseases

In some embodiments, disclosed herein are methods and compositions for the detection of Huanglongbing (HLB)/Citrus greening disease in plants. In some aspects, disclosed herein are methods and compositions for the detection of HLB/citrus greening disease in citrus plants.

In some embodiments, disclosed herein are methods and compositions for the detection of Citrus tristeza virus (CTV) in plants. In some aspects, disclosed herein are methods and compositions for the detection of CTV in citrus plants.

### Volatile Compounds

In some embodiments, the present disclosure relates to volatile compounds (VOCs). As used herein, “volatile compounds” includes any kind of “volatile compound”, including “induced volatile compounds” (IVOCs) and “biogenic volatile organic compounds” (BVOCs).

Plants emit into the atmosphere a significant amount of their fixed carbon as VOCs; the production of these VOCs is reflective of the internal physiological status of the host plant. For example, when plants are fed upon by insects or herbivores, their direct defense response is to release volatile organic compounds (Farmer, 2001). The emitted volatile compounds under stress response are often referred to as “induced VOCs” (IVOCs), which are released from the surface of plant leaves, fruits, and roots. This response is not only induced under biotic attack, but also by abiotic stresses as well, temporally changing the plant VOC profile. IVOCs play an important role in plant-to-plant communication (Baldwin et al. 2006; Bezemer and van Dam 2005; Rohloff and Bones 2005), herbivore defense (Glendinning et al. 2009; Kessler and Baldwin 2001; Runyon et al. 2006), and have been shown to aid resistance to biotic stress (Kishimoto et al. 2005; Park et al. 2007). Therefore, the composition of the emitted VOCs, which number in the thousands, contains important biochemical information of the underlying metabolic processes within the plant system (Dudareva et al. 2004; Sumner et al. 2003; Weckwerth 2008). The expressed VOCs can be collected and measured using analytical methods, providing a momentary snapshot of the plant health status. Measurement of the VOCs is therefore an attractive avenue as a non-invasive and rapid way of monitoring of physiological processes in plants, including: flowering (Müller et al. 2002), ripening (Herrmann et al. 2002), maturing (Rapparini et al. 2001), stress (Karl et al. 2008; Lee et al. 2009; Loreto et al. 2006), and disease state (Paolini et al. 2008).

### Methods of Capturing and Analyzing VOCs

In some embodiments, provided herein are methods for capturing VOCs from plants. As provided herein, plant VOC sampling can be performed in situ from whole plants, fruits, and leaves, or directly from detached plant tissues (Tholl et al. 2006).

In some aspects, the emitted VOCs can be collected onto solid adsorbents positioned proximally to whole plants, or collected onto sorbents using a vacuum system to sample large volumes of air from plants under field conditions. In one aspect, a commonly utilized technique employs a direct static headspace sampling approach, whereby the plant VOCs are collected/adsorbed onto functionalized surfaces termed solid phase microextraction (SPME) fibers. The collected volatiles are then thermally desorbed and the volatiles are introduced into a GC/MS system for chemical analysis (Stewart-Jones and Poppy 2006); this sampling methodology has been applied to profile both fresh and dried plant samples (Zini et al. 2002).

In some embodiments, provided herein are methods for the analysis of VOCs. Due to the large number of VOCs emitted by plants, a number of analytical techniques may be used in parallel to gather a global VOC fingerprint from various plant systems (Goff and Klee 2006).

### GC/MS

In one aspect, gas chromatography mass spectrometry (GC/MS) is used to analyze plant VOCs (Lytovchenko et al. 2009). Gas chromatography mass spectrometry is well developed analytical separation and detection technique, in which complex sample mixture is fractionated into simpler components through chromatographic separation and the eluted component is linearly introduced into the MS for detection and quantification. Furthermore GC/MS is ideally suited to the analysis of low molecular weight organic compounds such as VOCs, generating atomic and structural information of molecular compounds present within the sample. Sample introduction technique such as analytical thermal desorption (TD) have been hyphenated with GC/MS utilized with Tenax-TA and PDMS membrane for the sampling of VOC for non invasive analysis of biological samples (Yun).

In other aspects, nuclear magnetic resonance (NMR) or liquid chromatography mass spectrometry (LC/MS) may be used to analyze plant VOCs.

In some aspects, a portable detection device is used for detection of the biomarkers of interest. If the device of choice is portable, in-situ analysis can be possible. Portable detection devices include, without limitation, ion mobility spectrometry (IMS), differential mobility spectroscopy (DMS)/field asymmetric ion mobility spectrometry (FAIMS), and GC technology based units.

### DMS and GC/DMS

In one aspect, differential mobility spectrometry (DMS) is used to analyze plant VOCs. DMS is a gas phase separation and detection technique; it operates by exploiting the non-linear behavior of charged ions at rapidly alternating high and low electric fields over short macromolecular distances to induce separation and subsequent detection (Krebs et al 2005). Its ability to operate at ambient pressure with sensitivity at the ppb level (Eiceman et al 2004), its low power consumption, and its small size and potential for further miniaturization, enable DMS to be particularly well suited for the analysis for gaseous samples and in-field analysis of VOCs. In some aspects, DMS is coupled with (hyphenated with) gas chromatography (GC), in order to achieve additional chromatographic separation.

DMS has been extensively applied to the characterization of bacterial samples (Prasad et al. 2007; Schmidt et al. 2004). In addition, it has also been applied to the study of viruses (Ayer et al. 2008). DMS has been successfully applied to the analysis of VOCs from proliferating bacterial samples (Shnayderman et al. 2005), carbonized fire debris remains and jet fuel ((Lu and Harrington 2007; Rearden et al. 2007) for discrimination applications. GC/DMS has been applied to characterize and distinguish volatile compounds emitted from peel sections of normal healthy citrus fruit and those infected with citrus “puff” disorder (Zhao et al. 2009).

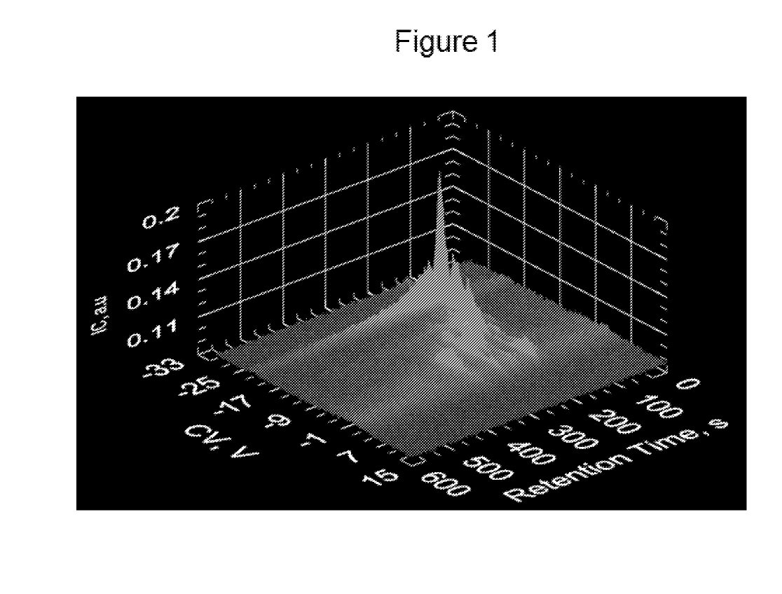

In one embodiment, a method is provided herein of using both GC and DMS detection for analysis of VOCs from biological samples. In some aspects, the combination of GC with DMS increases the diagnostic capacity of DMS. In a GC/DMS experiment, each chemical can be separated and characterized by its respective compensation voltage (CV)s and retention times, both indicative of a particular chemical species. A GC/DMS plot provides snapshot of volatile compounds emitted by a plant that can be used as a chemical signature (FIG. 1).

In some embodiments, methods of performing GC/DMS analysis are provided herein. In one aspect, each GC/DMS sample is characterized with a three dimensional data structure composed of retention time, compensation voltage, and the corresponding signal intensity. To make use of all the information in the 3-D data structure, original data may be kept without summarizing the signal across either retention time or compensation voltage. Principal component analysis (PCA) may be applied to the 3-D data to preliminarily visualize the distribution of samples from different groups. Based on the PCA results, next steps can be designed to explore the data.

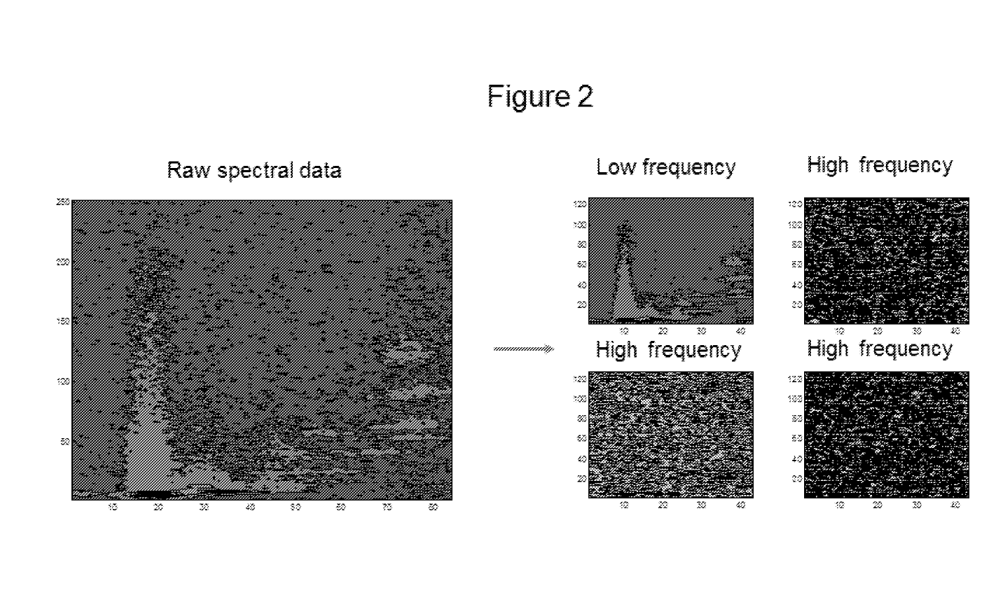

In some aspects, wavelet transformation is used to concentrate the major information into a low frequency domain, while removing the majority of noisy content into a high frequency domain (FIG. 2). Based upon the wavelet coefficient selection strategies, the pertinent coefficients may be retained for further analysis. In some aspects, multivariate analysis methods including linear approaches like Principal Component Analysis (PCA) and Partial Least Square (PLS) and nonlinear approaches like support vector machine can be employed to both visually and quantitatively examine separation between groups.

Calibrating a VOC Detection Device Against Compounds from a Biomarkers Library

In some aspects, a VOC detection device can be trained and calibrated using standards of compounds from a biomarkers library. A trained device may be capable of distinguishing a chemical of interest against complex background and in very low concentrations (for example, the detection limits of the DMS/FAIMS based devices can be as low as a few ppb).

In some aspects, the limits of detection can be further improved if the compounds of interest are pre-concentrated using absorptive membranes or other pre-concentrators and/or background removal means.

### Gene Expression

In some embodiments, the present disclosure relates to gene expression in plants. As is well known to one of skill in the art, in an organism, genes are encoded as DNA. Typically, for a gene to be expressed, the DNA encoding the gene is transcribed into mRNA, which then is translated into protein. The transcription of a gene into mRNA is referred to as “gene expression”. At the level of the genome, all of the mRNA collectively are referred to as the “transcriptome”, much as all of the DNA in an organism is referred to as its “genome”.

### Methods of Analyzing Gene Expression

Methods for analyzing gene expression are well known in the art. Methods include, for example, northern blotting, real-time PCR, and microarrays and RNAseq (whole transcriptome sequencing using next generation DNA sequencing technologies). A complete disease response to any specific pathogen or pest is represented in the complexity of the RNA population, including both coding (mRNA) and noncoding (small RNA) sequences. This can now be analyzed to an unprecedented depth using new, next-generation DNA sequencing methods (NGS) which reveal very rare mRNA, splice variants, allelic variants, and SNPs. This technology, already applied in plants (Navarro et al., 2009; Donaire et al., 2009) assumes extensive bioinformatics knowledge of the organism investigated. For plant species that lack whole-genome sequence information, an extensive EST database can be used instead. Transcriptomic data obtained are usually confirmed with qRT-PCR analysis or integrated with proteomic and metabolomic analysis. In addition, analysis of the deep transcriptome profile using biological network theory can help define gene regulatory networks and identify key disease-specific biomarkers.

In one aspect, a microarray technology for rapid, hybridization-based nucleic acid detection, as described in Carter and Cary 2007 and Cary 2007, is used for gene expression analysis. This integrated, sample-to-answer nucleic acid device may be used, for example, to identify expression of genes of interest in plants directly in an orchard. This device is also amenable to field use by untrained personnel, and can be realized using low cost lateral flow chromatography technology.

In another aspect gene expression can be evaluated using quantitative real time PCR (qRT-PCR), a technique already set up for pathogen detection. mRNA may be extracted from a sample and evaluating the level of expression of specific mRNA that serve as biomarkers for a particular disease. In one aspect, provided herein are disease specific gene expression-based biomarkers that can be detected to make a disease specific diagnosis using either qRT-PCR or field adaptable microarrays as indicated above.

### Plants

In some embodiments, the present disclosure relates to disease detection in plants. In some aspects, the present disclosure relates to disease detection in citrus plants. The methods, compositions, and devices provided herein may be used for example and without limitation, for detection of diseases of plants of the following types: citrus (sweet orange, including Washington Navel orange, Valencia orange, and Hamlin orange varietals, mandarin, Clementine, lemon, lime, and grapefruit), potato, and tomato.

### Methods of Disease Detection

In some embodiments, provided herein are methods for the detection of disease in plants. In some embodiments, provided herein are methods for the detection of disease in plants by analysis of VOCs from plants. In some embodiments, provided herein are methods for the detection of disease in plants by analysis of gene expression in plants.

### Methods of Disease Detection by VOC Analysis

In some embodiments, provided herein are methods for disease detection in plants by VOC analysis.

In some aspects, the present disclosure relates to a method for determining the presence of disease in plant material, such as whole plants, leaf material, fruits, berries, flowers, scions, flower organs, root stock, seeds, bulbs, algae, mosses and tubers of plants, by monitoring VOCs released by the plant.

In one aspect, the disclosure relates to a method wherein a disease is detected in a plant by VOC analysis. In a method of detecting disease in a plant by VOC analysis, a sample of VOCs released by the plant being tested for disease is obtained. The sample of VOCs released by the plant is then analyzed by one or more methods, in order to determine the identity and/or quantity of one or more VOCs present in the sample. The identity and/or quantity of VOCs present in the sample is then compared to VOC values from healthy and/or infected plants, in order to determine whether the plant being tested has a disease. In some aspects, the VOC values from healthy and/or infected plants are known before the time of the VOC analysis of the test plant, and the VOC values from the test plant are compared to predetermined values of VOCs that are correlated with healthy or diseased plants, in order to determine the disease status of the test plant. In some aspects, the VOC values from healthy and/or infected plants are determined at the same time or later than the VOC analysis of the test plant, and the VOC values from the test plant are compared with VOC values from healthy and/or infected plants once the values of VOCs that are correlated with healthy or diseased plants are known, in order to determine the disease status of the test plant.

In one aspect, the disclosure relates to a method wherein VOC profiles are mapped beforehand using appropriate analytical method, such as gas chromatography and mass spectrometry (GC/MS) and/or gas chromatography/differential mobility spectrometry (GC/DMS), and the VOC signatures indicative of the presence of a particular disease are identified. For non-in situ analysis, VOCs may be collected using specific adsorptive surfaces such as Solid Phase Microextraction (SPME) and Twister devices.

In some aspects, analysis of VOCs adsorbed on SPME fibers may be performed using GC/MS. Since distribution and/or composition of VOCs is altered by the presence of pathogen, the GC/MS profile can serve as signature for pathogen presence or absence. However, since MS allows for chemical identification, it may be advantageous to only select the statistically significant GC peaks of VOCs that are descriptive of the plant's health status. Any robust peak selection algorithm may be used to achieve this. The mass spectra associated with these peaks can be used to establish chemical identity of the volatiles of interest. An appropriate MS structure analysis approach, such as Electron Ionization (EI)/Chemical Ionization (CI) combination, MSⁿ etc. can be used.

In one aspect, the present disclosure relates to a method of in-field measurements of plant VOCs using an appropriate field sensor device for measuring the chemical signature of plant material, both in vivo and in vitro, when appropriate. Such measurements can be performed to detect VOC signature associated with a particular disease and to detect pathogen presence and identity based on previously assembled VOC libraries. Appropriate data mining approaches can be applied. Compounds from a database of pathogen biomarkers assembled prior to the in-field detection using GC/MS and/or other appropriate analytical methods of choice may be used for instrument training/calibration.

In one aspect, provided herein is a hyphenated analytical system to monitor and measure the volatile organic compounds emitted by citrus plant varietals for the disease detection in plants. In some aspects, VOC based sensors may be used to detect host plant response to pathogenic infection and to monitor the disease state of plants through in-field sampling of plants, looking for differences within the emitted VOC signature pre- and post-infection. In some aspects, an understanding of the baseline variability of background VOCs that emanate from citrus tree leaves may be obtained. In some aspects, analysis may be performed to determine how much diversity exists between commonly cultivated citrus varietals, as this may impact any library generation of disease-specific VOCs.

In some aspects, different varietals of the same species of citrus plant have similar but distinct expression of VOC profiles, and by analyzing the VOCs emitted from a plant, this signature may be used for discrimination between two different but closely related citrus varietals. In some aspects, using both GC/MS and GC/DMS detection by correlating the output between the two detection techniques may be used to establish VOC signatures of citrus volatiles for DMS.

In some aspects, provided herein is a method of classifying the health status of citrus, as well as other plants, based upon their released volatile metabolites, via identifying and measuring chemical signatures that are indicative of a particular disease, such as HLB or CTV in citrus. In some aspects, the classification procedure involves two major steps: 1) mapping of the VOC distribution indicative of the presence of certain pathogens; and 2) in-field measurements of the VOC signature and comparison to previously mapped VOC profiles in order to determine health status of the plant. In addition, aligning of responses of multiple analytical methods may be performed.

### Mapping of the VOC Profiles of Plants

The most comprehensive analysis of the VOCs can be carried out using non-portable analytical instrumentation such as GC/MS. The in situ collection of samples is possible using designated specific absorptive surfaces such solid-phase micro extraction (SPME) fibers or other solid sorbent phases (e.g. Twister). In some aspects, analysis will take into account that the volatile compounds may vary according to age, type of leaf and season of the year, among other factors.

Generally, it is desirable to minimize the number of potential variables in the experiment. The choice of fibers allows fine-tuning the range of compounds collected for further investigation; the greater range of compounds can be collected using a variety of fiber types. In one aspect, SPME fibers having a Carboxen/Polydimethylsiloxane (CAR/PDMS) polymer coating are used to collect volatiles emitted by citrus plants.

In one aspect, a VOC sampling procedure is performed as follows. Prior to the analysis by GC/MS, the fibers or sorbent phases are conditioned to remove any starting-point adsorbed chemicals from background environmental chemicals, as recommended by the manufacturer. For initial sampling, the fibers are positioned near the surface of the leaf in an aluminum holder to protect the fragile tip. To limit the effects of the diurnal cycle on leaf VOCs, sampling may be carried out at specific time points in the day. The exposure time depends on the efficiency of VOC production by plants. One factor to take consider may be the ambient temperature; in some aspects, the greatest VOC production may occur in the 60-75 F range. Exposure time may vary depending on the conditions. In some aspects, exposure time may be for around 1, 2, 3, 4, 5, 6, 7, 8, 9, 10, 11, or 12 hours. In some aspects, exposure time may be around 6 hours at optimal VOC production conditions, and around 12 hours at non-optimal VOC production conditions (e.g. overnight during cool time of the year). After sampling, the fibers can be submitted for biochemical analysis.

In some aspects, correlation of the data from different analytical techniques may be performed. The compound signatures from the GC/DMS can be correlated to GC/MS data, so that chemical compound identification can be carried out in both data sets. The individual peaks on the GC/MS chromatogram can be correlated with the output spectra of the DMS. Aligning spectra using appropriate reference compounds will allow correlation of a peak in the GC/MS signal domain with the matching equivalent peak in the GC/DMS domain. Such matching may allow establishing a chemical library database for a DMS sensor. In some aspects, it is possible to locate important VOC metabolite biomarkers in the GC/DMS signal space that are not represented in the GC/ITMS data, or vice versa, due to differing sensitivities to certain chemicals of the two detectors.

In some aspects, the classification accuracy using GC/DMS device may be further improved if, instead of total analytical space, only signals from pathogen biomarkers are considered. The detection device can be trained and calibrated using standards of the compounds from the biomarkers library. The trained device may be capable of distinguishing the chemicals of interest against complex background and in very low concentrations (the detection limits of the DMS/FAIMS based devices can be as low as a few ppb). The limits of detection can be further improved if the compounds of interest are pre-concentrated using absorptive membranes or other pre-concentrators and/or background removal means. When a chemical compound associated with the particular disease is detected, the sensor will provide positive output. If a number of chemicals from the data base associated with certain disease are detected, the positive output will have greater validity (lower chance of false positive). It can be left to the discretion of the operator which validity threshold is optimal under certain conditions to consider a plant as pathogen-free or infected. Adjustments can be made for a particular plant variety, orchard, time of the day, season etc., as deemed appropriate.

### Detection of HLB in Plants by VOC Analysis

In some embodiments, provided herein are methods for the detection of HLB/citrus greening disease in plants by analysis of VOCs. VOCs may be obtained from plants by methods described herein, and VOCs may be analyzed as described herein, in order to identify the chemicals represented in diseased and control plants. Selected VOCs can be used for HLB pathogen detection as well as correlation of the VOC emission with the metabolic changes in plants during the course of HLB infection.

In some aspects, the present disclosure relates to methods for determining the volatile compounds emitted from leaves of citrus trees associated with HLB disease. The disclosure also relates to methods wherein the VOC profiles are recorded by application of gas chromatography and mass spectrometry (GC/MS) and the biomarkers indicative of the presence of the HLB pathogen are identified. VOCs may be collected using specific adsorptive surfaces such as Solid Phase Microextraction (SPME) and Twister devices.

In some aspects, the present disclosure also relates to the application of appropriate data mining methods to establish the differences in gas chromatograms of healthy and HLB-diseased plants. The disclosure particularly relates to the identity of the HLB biomarkers, which include but are not limited to, the following compounds: Carbon dioxide, Propane, 2-methyl-Pentane, o-Xylene, Tridecane (C13H28), 2-ethyl-1,4-dimethyl-Benzene, 1-methyl-4-(1-methylethenyl)-Benzene; 2,2,3,4-tetramethyl-Pentane; Hydrocarbon, e.g. Pentadecane (C15H32) etc.

### Detection of CTV in Plants by VOC Analysis

In some embodiments, provided herein are methods for the detection of Citrus tristeza virus (CTV) in plants by analysis of VOCs. VOCs may be obtained from plants by methods described herein, and VOCs may be analyzed as described herein, in order to identify the chemicals represented in diseased and control plants. Selected VOCs can be used for CTV pathogen detection as well as correlation of the VOC emission with the metabolic changes in plants during the course of CTV infection.

VOC analysis may be used for the detection of CTV in citrus varietals, and in some aspects, it may be used for in-field, real time monitoring of plant health. In one aspect, VOC analysis is advantageous because it is non-invasive. Biogenic volatile organic compounds (BVOC) are a form of VOCs generated by all living organisms and in particular plants for the purpose and maintenance, growth and function. BVOCs are also released as a part of a stress response due to abiotic/biotic stress (water and water stress), zinc and nutrimental deficiencies. BVOCs may also be referred to as “induced VOC” (IVOC). The BVOC/IVOC profile emitted from leaves of citrus varietals may be significantly altered as a result of post infection responses to CTV.

In some aspects, in-field VOC sampling methods of Citrus varietals using twisters and static head sampling with thermal desorption gas chromatography time of flight mass spectrometry (GC/TOE-MS) analysis is used for the discrimination between healthy and CTV infected crops. In some aspects, VOC sampling methods provided herein are used to monitor plant health for CTV.

The disclosure particularly relates to the identity of the CTV biomarkers, which include but are not limited to, the following compounds: myrcene, carene (delta-3-), ocimene (e-beta-), hexadecanol, limonene, tetracosane, and bulnesene (alpha-).

### Methods of Disease Detection by Gene Expression Analysis

In some embodiments, provided herein are methods of disease detection in plants by gene expression analysis.

In one aspect, in a method of detecting disease in a plant by gene expression analysis, a sample of nucleic acids (e.g. RNA) expressed by the plant being tested for disease is obtained. The sample of nucleic acids expressed by the plant is then analyzed by one or more methods, in order to determine the identity and/or quantity of one or more nucleic acids present in the sample. The identity and/or quantity of nucleic acids present in the sample is then compared to gene expression values from healthy and/or infected plants, in order to determine whether the plant being tested has a disease. In some aspects, the gene expression values from healthy and/or infected plants are known before the time of the analysis of the gene expression analysis of the test plant, and the gene expression values from the test plant are compared to predetermined values of gene expression that are correlated with healthy or diseased plants, in order to determine the disease status of the test plant. In some aspects, the gene expression values from healthy and/or infected plants are determined at the same time or later than the gene expression analysis of the test plant, and the gene expression values from the test plant are compared with gene expression values from healthy and/or infected plants once the gene expression values that are correlated with healthy or diseased plants are known, in order to determine the disease status of the test plant.

In some aspects, disease detection in plants by gene expression analysis is based on the analysis of the early host responses and the identification of early regulated genes in specific highly physiological active tissues such as leaves and fruit peel tissues. In some aspects, these genes are used in multiple qRT-PCR assays focusing on host responses, which may be used to complement pathogen-directed disease tests, or to allow early detections at asymptomatic stage when pathogen titers are below the threshold of sensitivity of another instrument for disease detection.

Disease and the environmental conditions affect short and long distance signaling mechanisms in the host plants. Infection that occurs in leaf tissues may induce a transcriptomic response in other tissues (such as fruits), where the signals of infection are amplified by the induction of host response genes. A pathogen-induced gene may be transcripted in hundreds or thousands of RNA molecules, while pathogen DNA might be present in only few copies and may be below sensitivity level of detection. The expression of each host response in genes may be tissue and/or developmental stage dependent. In certain aspects, particular plant tissue may have a gene expression pattern in response to pathogens that may be used as a sensor of a pathogen in a plant.

In some aspects, gene expression biomarkers may be used to improve disease management programs by clarifying the disease status of existing trees. In some aspects, analysis of gene expression in plants permits disease detection at an early, asymptomatic stage where limiting secondary infections in a plant is still practical. In addition, in some aspects, gene expression biomarkers may be used to validate potential therapeutic strategies as they become available and to screen for resistance in the citrus germplasm or validate transgenic approaches. In some aspects, by analyzing citrus gene expression in response to pathogens, citrus germplasm can be screened for resistant cultivars to include in genetic improvement programs using traditional or biotechnological approaches.

### Identifying Relationships Between Gene Expression and VOCs

In some embodiments, provided herein are methods for the correlation of plant gene expression with plant VOC release in response to pathogens and/or environmental conditions.

In some aspects, gene expression host biomarkers of early infection or environmental response can be readily integrated into current and future disease diagnostic technologies and platforms like PCR, Lateral Flow Microarray (LFM), Differential Mobility Spectrometer (DMS) and GC/MS for the co-detection of specific gene transcripts or volatiles, thereby greatly increasing the scope, range, and/or accuracy of disease detection in plants.

The present disclosure also relates to correlation of different analytical methods, i.e. to the aligning of the instrumental response of different analytical methods used in conjunction.

### EXAMPLES

The following Examples are merely illustrative and are not meant to limit any aspects of the present disclosure in any way.

### Example 1

### Gene Expression and HLB

An appropriate experimental design was developed to examine host response and clearly identify response biomarkers regulated by HLB at different infection stages. Four types of tissues were analyzed for fruits and leaves at young and mature stage. The first two categories were symptomatic and asymptomatic samples (peel and leaves) from infected “Valencia” sweet orange (C. sinensis L. Osb.) trees located at the USHRL-USDA Farm in Fort Pierce (St. Lucie County, Fla.). Trees were analyzed by PCR for the presence of CaLas using petioles from 4-6 leaves collected from different areas in the canopy. The third category were fruit from PCR-negative, apparently healthy trees at the same location. The fourth category was composed of fruit from healthy ‘Valencia’ trees in a disease-free location at the Citrus Research and Education Center (Lake Alfred, Fla.). RNA was extracted using phenol/chloroform/isoamylalcohol (25:24:1) extraction and then purified using RNeasy MinElute Cleanup kit (Qiagen, Valencia, Calif.) according to the manufacturer's instructions. The RNA was used to perform a deep transcriptome analysis using Illumina Genome Analyzer II following manufacture instructions for cDNA libraries construction and sequence run analysis. The quality of each library was determined using a bioanalyzer (BioRad, Hercules, Calif.). Each library was run as an independent lane to obtain read lengths of up to 85 base pairs per end of each cDNA molecule sequenced. This type of procedure is referred to as RNAseq.

Raw data were processed and assembled using Velvet software. The alignment of the individual reads and contigs to the Citrus sinensis unigene set (15,808 sequences; NCBI Unigene Build #11, Apr. 20, 2009) was performed using BWA (Li and Durbin, 2009).

Functional analysis of the transcriptomic data was performed using Mapman and Blast2GO software to categorize the differentially regulated genes in pathways and networks and understand the major changes in the cell metabolism induced at different stages of disease. After statistical analysis, pathways that were most affected by the disease were identified, and within them, key genes were chosen that seemed to be highly regulated at asymptomatic stages. These genes were analyzed with qRT-PCR analysis to validate their pattern of expression and verify if they can work as early indicators of infection in plants where HLB pathogen could not yet be detected by other detection methods.

Differentially regulated genes by CaLas infection in fruits between the 6 pairwise comparisons (control, apparently healthy, asymptomatic and symptomatic stages), represented by the Citrus sinensis unigenes (present in NCBI), are shown in Tables 1-6. Relating to leaves, HLB-differentially regulated genes at apparently healthy, asymptomatic and symptomatic stage were shown in Tables 9-14.

The following genes were shown to be statistically differentially expressed in fruit tissues at asymptomatic stage of the disease using Taqman Real Time PCR and they can be considered as host HLB-biomarkers: GH3.1 (upregulated) (S22545043); GH3.4 (upregulated) (S44237769); KA02 (downregulated) (S44303609); Salicylic Acid Methyl transferase (upregulated) (S44277040); WRKY70 (upregulated) (S44288591); MYB-related TF (upregulated) (S44256583); U-box (S22566824); HSP82 (downregulated) (S44237646); Invertase (upregulated) (S35152777); terpene synthase cyclase (downregulated) (S22583829); NN Lipid transfer protein (LTP) (upregulated) (S44279331); acidic cellulase 8 (downregulated) (S22606212); omega-6-FAD (downregulated) (S44244604).

Related to leaf analysis (immature and mature stages), several differentially regulated genes at asymptomatic stage were identified, usable as biomarkers in qRT-PCR analysis. At immature stage these genes are: Acidic cellulose (S22606212); Terpene synthase 1 (S44285742); ERTF2 (S44250648); 12-oxo-phytodienoate reductase (S34125138); lypoxygenase 2 (S34124539); NNLTP (NCBI accession number: EY754661.1); Beta-amylase (S44303510); Expansin 3 (S22533016), glucose-phosphate-transporter2 (S22591828, S22591828, S44257732, S22591828). At mature stage these candidate biomarkers were: ENT-kaurenoic acid hydroxylase 2 (S44251582); Expansin3 (S22533016), Alpha-amylase (S44224848); WRKY70 (S44225142, S44227900, S44288591).

### Example 2

### HLB Infection—GC/MS of VOCs

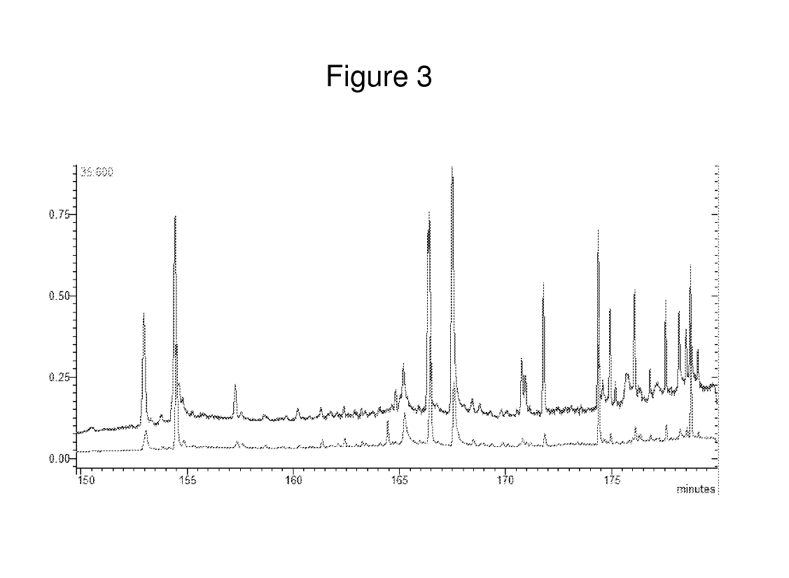

The laboratory-based traditional gas chromatography mass spectrometry (GC/MS) analysis will allow for identification of differences in VOCs production due to pathogen infection and identify specific “biomarker” compounds using the MS data. The analysis of VOCs adsorbed on SPME fibers was performed using GC/MS (FIG. 3). Upon heating, the adsorbed chemicals can be desorbed to be introduced into the GC/MS instrument and analyzed. Since distribution of VOCs is altered by the presence of pathogen, the GC/MS profile can reveal chemical compounds associated with pathogen presence or absence. Any robust peak selection algorithm can be used to select statistically significant peaks that discriminate healthy and infected plants. In our example case, the resultant data were further processed as follows.

To reduce the dimension of chromatography data and resist potential disturbance from possible noise and mild signal misalignment, the auto-regression (AR) model was applied to extract the features from each chromatogram. [Zhao 2009, Zhao 2008] A p-order AR model can be expressed by the following equation (1): (please see equation from cited manuscripts) (1) where, x(n) is the signal point of a data series, ai is the AR coefficients, p is the model order, and en is the estimation error. The AR model aims to predict the nth value x(n) from its previous p values: x(n−1), x (n−2), . . . , x (n−p), using the coefficients ai (i=1, . . . , p, p: model order). The goal of an AR modeling process is to estimate the AR coefficients that can fit the original data through an optimization process. Using this model, each chromatography profile can be characterized with a p-dimensional feature vector (a1, a2 . . . ap). As the first step in identifying the discriminatory peaks, the chromatographic peaks were located in each profile based upon the peak abundance and the mean-value crossing rate within a predefined window. Setting a single side length of a neighboring range for point i to be k, point i was considered as a peak candidate if it had higher intensity than all the points within the range of [i−k, i+k]. To ensure that the selected peak would not be a noise signal, a mean-value crossing rate of signal points (•) was defined. If there is a peak within the range [i−k, i+k], the peak and its neighboring points need to be clearly above the mean value of the signals within this range. In other words, the signal points in this peak window do not vibrate around the mean value. In addition to the zero crossing ratio, we propose to introduce a criterion called “mean-value crossing rate” (η) to quantify the signal vibration around the mean value. The lower the mean-value crossing rate is, the more unlikely the observed signal would be noise. For the chromatograms of orange trees samples, the values of k=10 and η=50% were used. After detecting the peaks, Student's t-test was applied to the corresponding peak intensities. The peaks that presented a small p value (p<0.05) were considered as a statistically significant and potential biomarkers. To investigate the classification between control and infected plants, principal component analysis (PCA) and principal component regression (PCReg) respectively, was employed to visually and quantitatively examine the classification results. The leave-one-out strategy, which is a typical validation strategy for small sample sets, was coupled with PCReg to provide a quantitative estimate of the diagnosis accuracy. [Bullinger, 2008; Freitas, 2008]

The GC peaks that were identified as discriminating features of GC profiles for healthy and diseased batches are documented. The mass spectra associated with these peaks were used to establish chemical identity of the chemical compounds of interest. An appropriate MS structure analysis method, such as Electron Ionization (EI)/Chemical Ionization (CI) combination, MSⁿ, etc., can be used. The resulting list of chemicals can serve as the reference database for biomarkers detection for particular plant types. Such databases can be expanded to a variety of pathogens. The database can be further updated when new biomarkers are discovered. Also, the compounds that were found to result in insufficiently robust differentiation can be removed from the database.

In the present disclosure, it is suggested that the chemical compounds specific to Hamlin orange trees affected by HLB disease include, but are not limited to compounds: Carbon dioxide; Propane; 2-methyl-Pentane; o-Xylene; Tridecane (C13H28); 2-ethyl-1,4-dimethyl-Benzene; 1-methyl-4-(1-methylethenyl)-Benzene; 2,2,3,4-tetramethyl-Pentane; Hydrocarbon, e.g. Pentadecane (C15H32) etc. By monitoring these compounds, it may be possible to detect HLB-affected plants. It is likely that the biochemical pathways that result in specific VOCs are not known; however, measurement of these VOCs in conjunction with metabolites expression mapping may make it possible to diagnose plant infections that may help the grower decision making.

### Example 3

### HLB Infection—SPME GC/MS HLB (Florida)

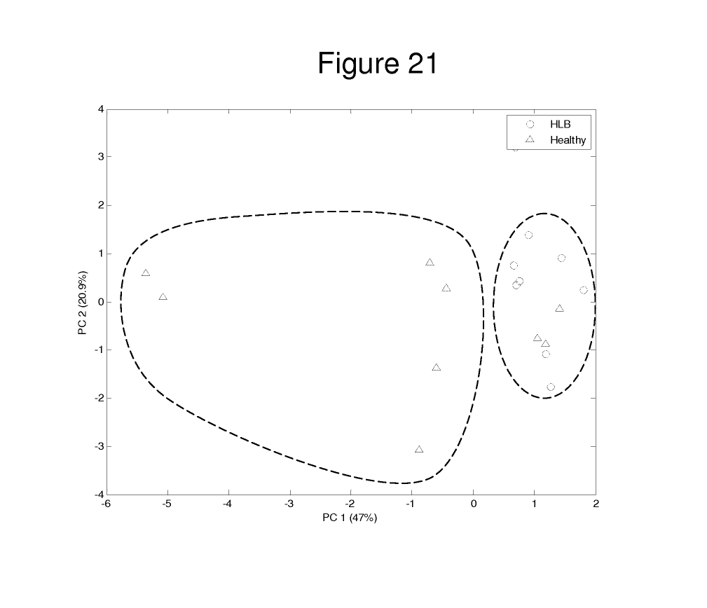

In this study, 9 distinguishable biomarkers were determined to yield a separation between HLB and healthy plants. From 18 collected samples (9 HLB-infected, and 9 healthy) we found 9 peaks that have a significant Student t-test result (p<0.1) (FIG. 21). 9 potential biomarkers that may be used to distinguish between HLB-infected and healthy plants were determined: Carbon dioxide; Propane; 2-methyl-Pentane; o-Xylene; Tridecane (C13H28); 2-ethyl-1,4-dimethyl-Benzene; 1-methyl-4-(1-methylethenyl)-Benzene; 2,2,3,4-tetramethyl-Pentane; Hydrocarbon, e.g. Pentadecane (C15H32) etc. Specifically, o-Xylene, Tridecane, 2-ethyl-1,4-dimethyl-Benzene, 1-methyl-4-(1-methylethenyl)-Benzene, 2,2,3,4-tetramethyl-Pentane, Pentadecane are up-regulated in HLB; Carbon dioxide, Propane, 2-methyl-Pentane, Tridecane are down-regulated in HLB-infected plants. Using a leave-one-out validation strategy, PLSR yields a classification accuracy of 83.33% (6/9 for healthy and 9/9 for HLB).

### Example 4

### Development of a Volatile Database

A database to analyze and interpret volatile profiles obtained from field and greenhouse samples using Twister-GC-TOF methodology was developed. A volatile BinBase database (vocBB) can be queried for spectra, compound identifiers or compound names through the public web query interface (http://eros.fiehnlab.ucdavis.edu:8080/binbase-compound/, choose ‘volatile’ when selecting the database).

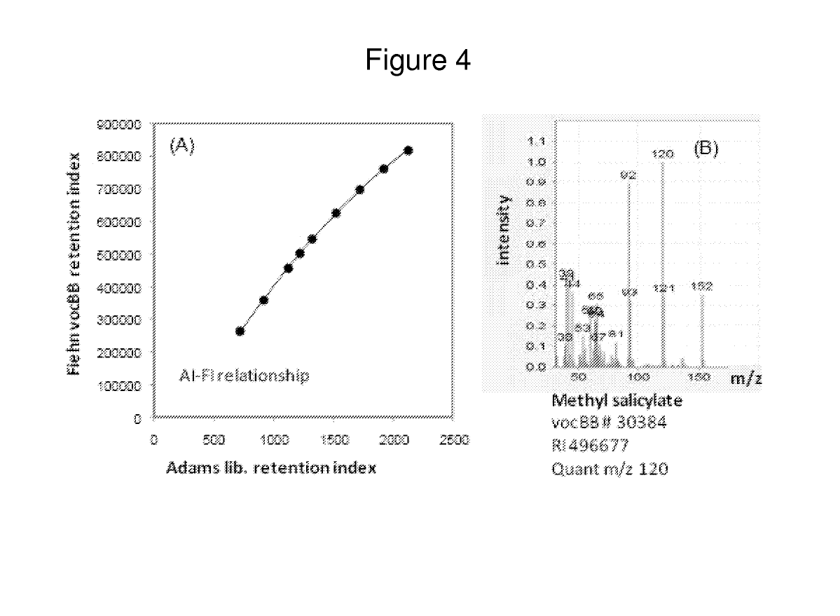

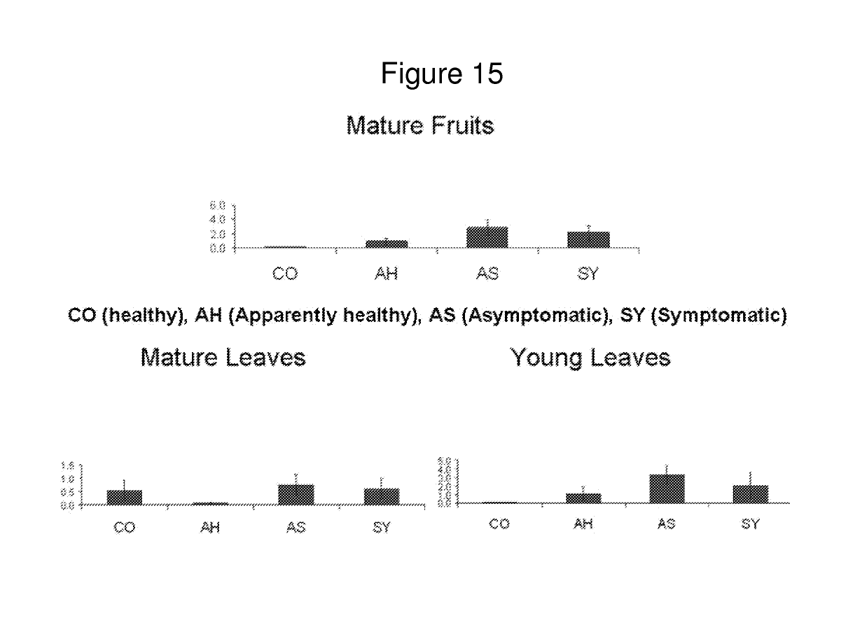

FIG. 4, panel (A), shows how volatile compounds were identified as genuine metabolites: first the commercial Adams volatile library that employs the classic alkane-base Kovats retention index was used, which was converted into our “Fiehn retention index” which is based on (more suitable) fatty acid methyl esters. All 2,000 Adams volatile spectra were converted and support the Fiehnlab vocBB in addition to authentic standards that were purchased. For example, the potential volatile compound methyl salicylate (see the induction of salicylic acid methyltransferase in response to HLB infection in mature fruits and young leaves (FIG. 15)) was positively detected in the over 2,100 volatile profiles that have been acquired so far and was annotated by matching mass spectra and retention indices by the Adams library, see FIG. 4, panel (B).

To date, the vocBinBase database has stored 1,465 valid volatile spectra, of which 183 were identified as chemical artifacts that originate, for example, from Twister coating or from plastic bag wrappings. Such artifact peaks are automatically excluded from data exports, i.e. cannot confound statistical analyses of citrus plant infection IVOC profiles. In addition, the vocBB features the possibility to annotate identified metabolites with database identifiers in order to straightforwardly integrate volatile profiles with genomic data via common enzyme and gene annotations. For example, methyl salicylate is stored in the KEGG biochemical pathways database as C12305 which links to the benzoic acid pathway map07110. While KEGG did not assign gene numbers to the formation or degradation of methyl salicylate, alternative databases confirm biological pathways, here: the MetaCyc database that links methyl salicylate to the Arabidopsis gene AtBSMT1 (At3g11480) via the TAIR AraCyc resource.

Therefore, the vocBB volatile databases have been constructed by allowing multiple database identifiers to be exported with volatile profiling datasets. All identified metabolites are curated in vocBB for these multiple cross-database numbers in order to simplify integration of volatile profiles with gene expression networks.

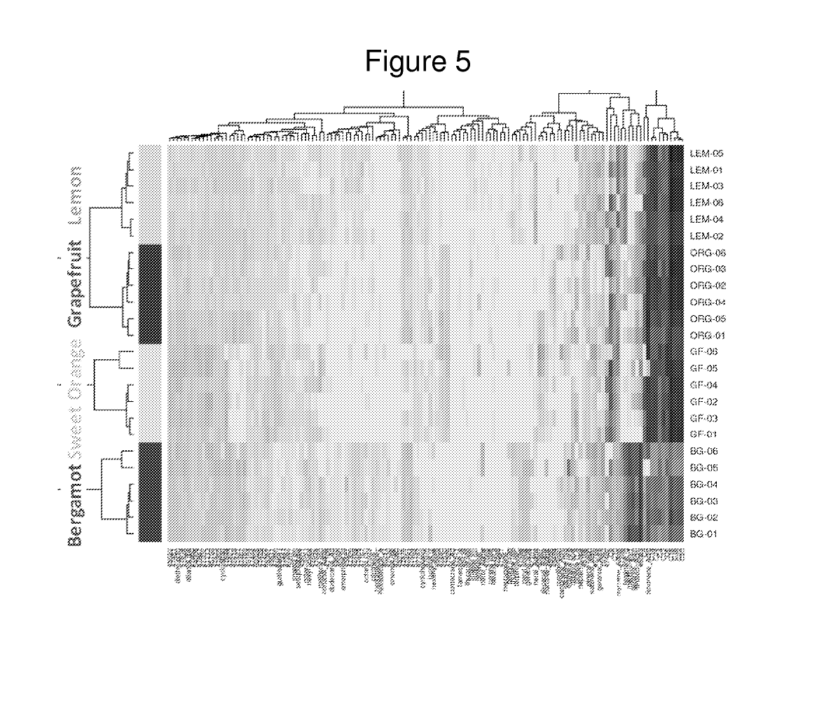

The vocBB database itself is constructed from actual Twister GC-TOF mass spectrometry profiles. As of July 2010, vocBB comprises 1.2 million mass spectra that were generated from 2,125 samples studying 18 species. A number of commercially available species reference standards, aka ‘essential oils’, have been used to increase the number of species and consequently, the number of genuine volatile metabolites that are stored in the database. For example, various citrus essential oils have been used as depositor samples into the database which also proves the functionality of the Twister GC-TOF technology in conjunction with database processing of the data. Volatile profiles of Bergamot, Sweet Orange, Grapefruit and Lemon generated many novel entries of volatile metabolites in the database (FIG. 5). Using simple hierarchical clustering of the profile data, all samples of these essential oils were correctly clustered without any wrong classification. This test provided evidence that the Twister GC-TOF is easily capable of detecting volatiles and using the intensities (blue to red colors in the cluster diagram) to classify sample origins. Thus, this study can be regarded as proof-of-principle for citrus volatiles. It also shows that citrus fruit oils could be used for HLB infection detection, as we have gained evidence that there are large gene expression differences in citrus fruits of HLB infected compared to apparently healthy and control trees.

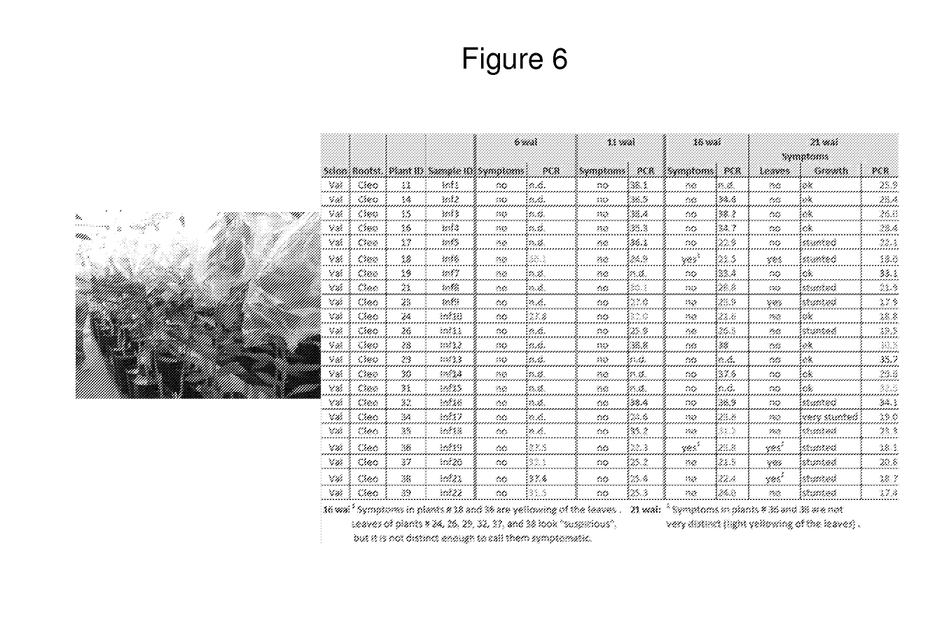

An HLB infection study has also been performed under controlled greenhouse conditions. FIG. 6 shows the experimental set up and records of HLB infection data. After inoculation of citrus plants with HLB (Cleopatra rootstocks×Valencia scions), volatile profiles were recorded for 22 infected plants and 10 healthy controls at 6, 11, 16 and 21 weeks. The development of infection was monitored by PCR and development of symptoms. Cleaned Twisters and cleaned bags were sent via mail to field locations in Florida; volatiles were then trapped on Twisters that were placed within enclosed citrus branches of infected and healthy control plants using in Reynolds™ oven bags. Sampling was performed starting at 10 AM for one hour exposure times, staggering each plant by 5 minutes. As negative controls, greenhouse air as well as air from empty bags was sampled by Twisters volatile adsorptions at each time point. Air temperature was 75° F., humidity 89.6%. Subsequently, Twisters were sent back via mail to UC Davis for analysis.

In total, 456 volatile compounds were detected in this study, of which 260 were retained as being detected at higher concentrations in bagged samples compared to the negative controls. Interestingly, a range of 191 volatile metabolites were detected in this study that were not present before in the vocBinBase database, again demonstrating the usefulness of a database approach compared to a target approach in which analytes would be pre-defined and subsequently screened. Some of these new, citrus-dependent metabolites were unambiguously identified using retention index and MS matching as given above: z-caryophyllene, benzyl benzoate, camphene, δ-3-carene, citronellal, 2,5-dimethoxy-p-cymene, diethylsuccinate, p-ethyl acetophenone, isopropyl tetradecanoate, cis-p-menth-2-en-1-ol, methyl decyl ketone, methyl geranate, neral, 2-octanol, α-pinene, cissabinene hydrate, sabinene, sesquisabinene, γ-terpinene, terpinolene, n-tetradecanol and α-thujene.

Overall, 79 metabolites could be identified in the controlled HLB-infection time course study and would thus be amenable for pathway analysis and comparison to transcriptomics data. The volatile profiling data were analyzed both with univariate statistics and multivariate tools. In principle, univariate methods are advantageous as results are easier to comprehend, and potential biomarkers can be used in a more straightforward way in potential field tests and validation studies. Unsurprisingly, the most important parameter that influenced volatile profiles in this study was the time course itself, as citrus trees were still young and actively developing with continuously maturing leaves.

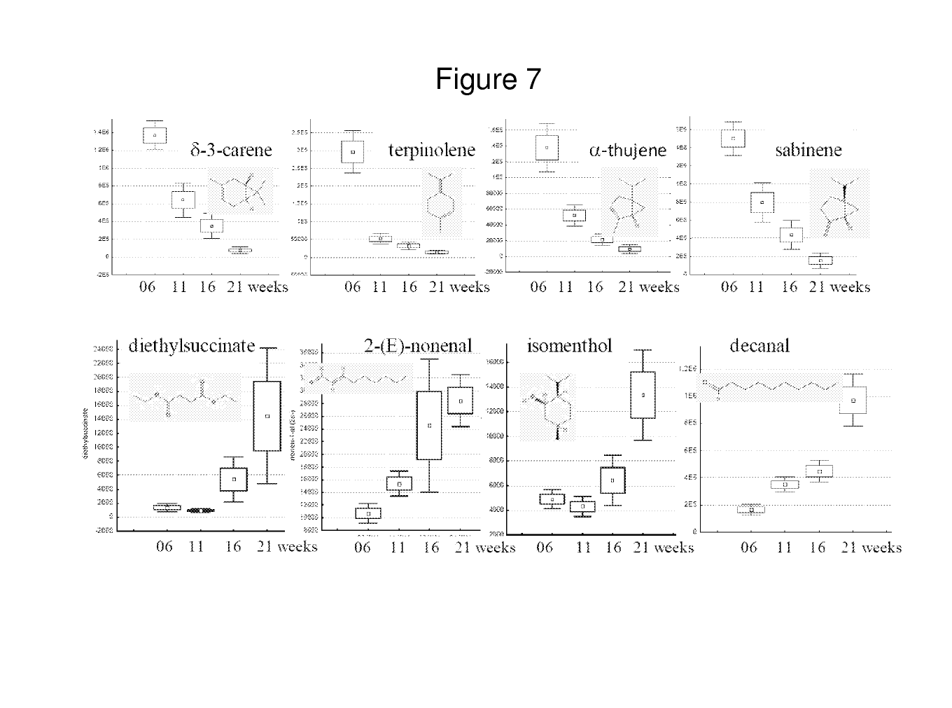

Consequently, it was determined that almost all volatile metabolites were affected by plant development, either by increasing, or by decreasing intensities, and some even by most intense emissions at 11-16 WAI. FIG. 7 shows exemplary temporal profiles integrating both healthy and infected plants. It becomes apparent that many terpenoids were decreasing in signal intensity, likely due to leaf maturation, which is reflected in the downregulation of gene expression terpenoid pathways in HLB-infected fruits. Other compounds were increased over time, also shown in FIG. 7.

Subsequently, volatile profiles were investigated for the impact of HLB infection compared to healthy controls. As evident by monitoring symptoms and PCR positive detection of HLB infection, initially few plants were detected PCR positive for HLB at 6 weeks after infection, whereas at 21 weeks, most inoculated plants tested PCR positive and actually showed visible disease symptoms as well as stunted growth. Similarly, there were initially very few volatile compounds that were significantly different at p<0.05 between healthy and infected plants, whereas 100 volatiles tested differently at 21 weeks after infection.

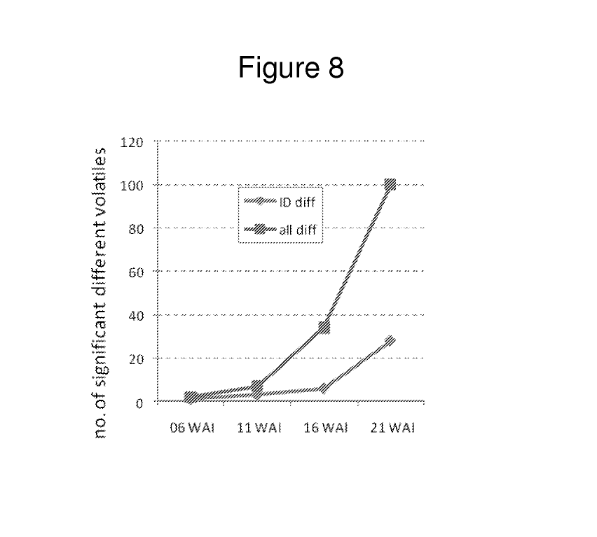

Surprisingly, most of these significant differences were suppressed in emissions (FIG. 8) while only a few compounds were increased. Unlike the results suggested by some gene expression studies, we did not find terpenoids increased after infection or even detect the presence of methylsalicylate. Methylsalicylate may work at the intracellular level as a messenger compound and may not be released into the atmosphere as a potential biomarker.

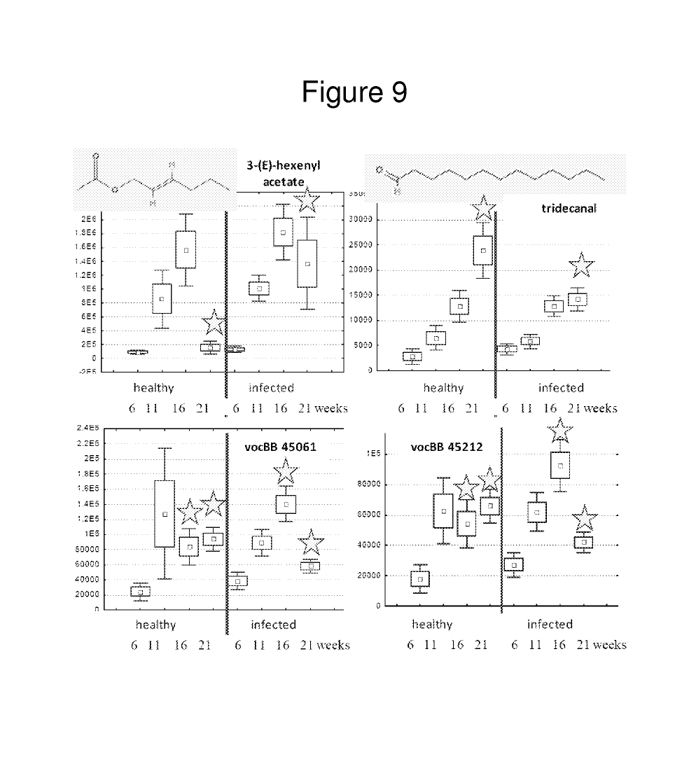

Instead, we have identified a range of potential volatile metabolite biomarkers for detection of HLB infections, some of which occur before symptoms are manifest. Examples are given in FIG. 9, showing hexenylacetate and tridecanal as two differentially regulated compounds under HLB infection at 21 weeks, whereas structurally yet unidentified metabolites may indicate HLB infection even at earlier time points (FIG. 9). The range of differences in metabolic genes (see Example 5 below) indicates that primary and secondary metabolites may be up-regulated even before volatile emissions.

### Example 5

### Correlation of Gene Expression Data and Volatiles

In this Example, a biological regulatory network was developed to correlate and visualize gene expression data obtained by deep transcriptome sequencing. These activities were accomplished by focusing on the discovery and validation of biomarkers for the early detection of HLB based on the analysis of the transcriptome of citrus.

We used previously published datasets of transcriptome analysis of leaves as well as those generated in our lab obtained from citrus fruit peel tissues using RNA-seq technology on an Illumina GA-II analyzer. The latter dataset has revealed the information described and validated below. This dataset contained 158,656 contigs that were compared to the NCBI Citrus sinensis unigene set that contained 15808 unique genes (this was done as we do not still have a reference citrus genome sequence).

Roughly half (45.7%) of these genes (15808) could be matched to our contigs with more than 90% of the mapped reads being assigned to 1 Citrus NCBI unigene. Once a contig is matched we use the total number of reads associated with that contig as the metric to measure expression of that gene. We made 6 pairwise comparisons between symptomatic, asymptomatic, apparently healthy and wild-type fruits to calculate the changes in the expression (log fold ratio) of individual genes. Of the 15808 genes that we examined we found that about 1156-1734 were differentially expressed (either up or down regulated) during various stages of HLB infection.

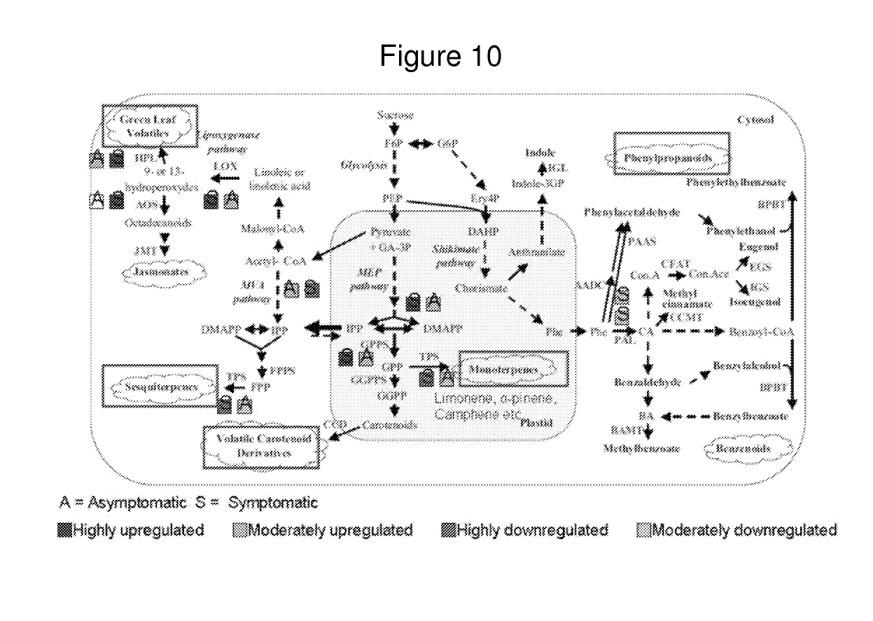

We then did a functional characterization of these genes using Pathexpress software (Conesa et al., 2005) and MapMan software. We observed that among the significantly regulated pathways, many involved pathways that lead to the biosynthesis of volatile (scent and aroma) compounds, and we have highlighted these pathways in FIG. 10 as they are regulated at asymptomatic and symptomatic stages in fruit. Transcriptional changes were observed in pathways that lead to the synthesis of terpenoid compounds and other important pathways. These include the non-mevalonate pathway or 2-C-methyl-D-erythritol 4-phosphate/1-deoxy-D-xylulose 5-phosphate pathway (MEP/DOXP pathway) of isoprenoid biosynthesis which takes place in plastids. Also the plastid 4-hydroxy-3-methylbut-2-en-1-yl diphosphate reductase (ISPH) and the geranylgeranylpirophosphate synthase 1 gene were upregulated in HLB-infected fruits and this might affect the carotenoid biosynthesis and the volatile compounds associated with this pathway. In the cytosol, HLB-regulated pathways included the biosynthesis of jasmonic acid, mevalonic acid-pathway that produces the sesquiterpenes and sterols.

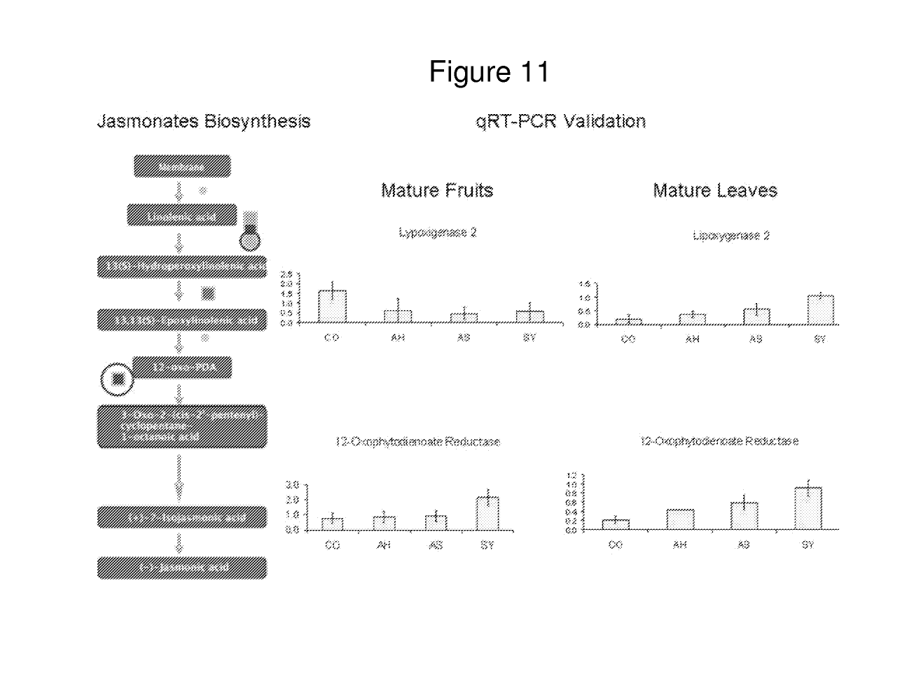

Transcriptomic analysis showed that genes encoding lipoxygenases, allene oxide synthase and 12-oxophytodienoate reductase were induced at early and late stage of disease (FIG. 11). These results were validated using qRT-PCR analysis choosing two genes: lypoxigenase 2 and 12-oxophytodienoate reductase.

Quantitative real time analysis in mature fruits confirmed the pattern of expression determined by deep transcriptome profiling. Interestingly, in mature leaves both genes were induced at symptomatic stage and this demonstrated that the Jasmonic acid-mediated defense response is induced by HLB infection also in leaf tissues.

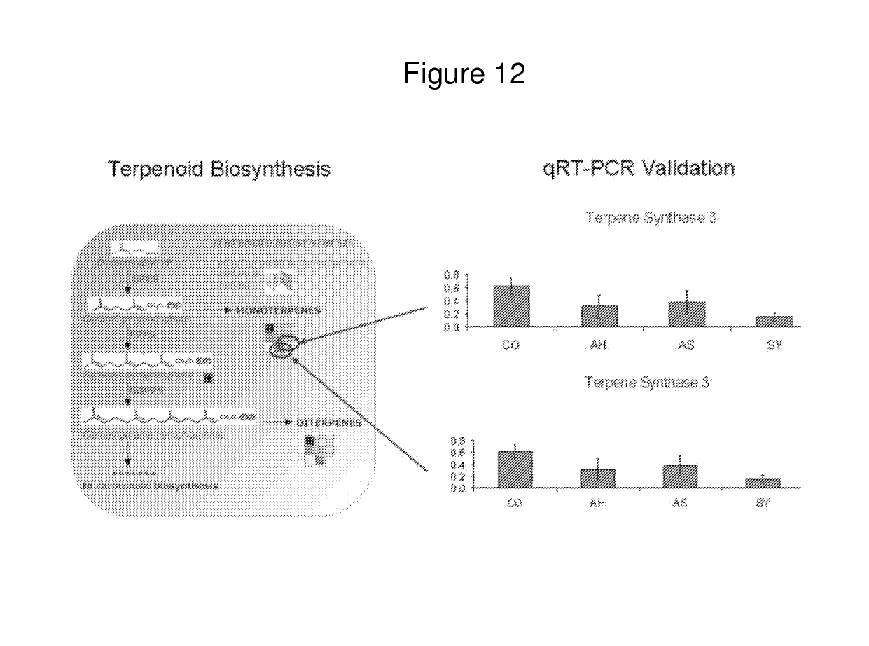

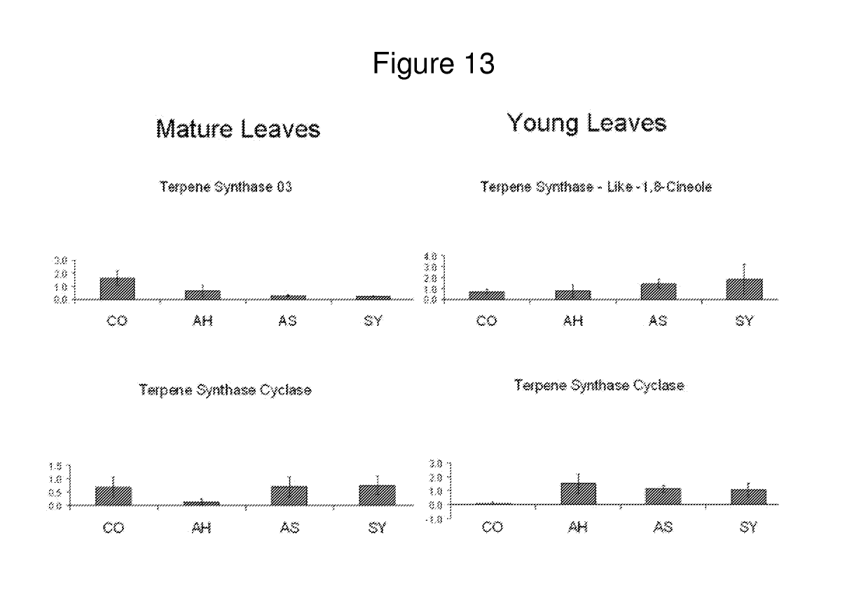

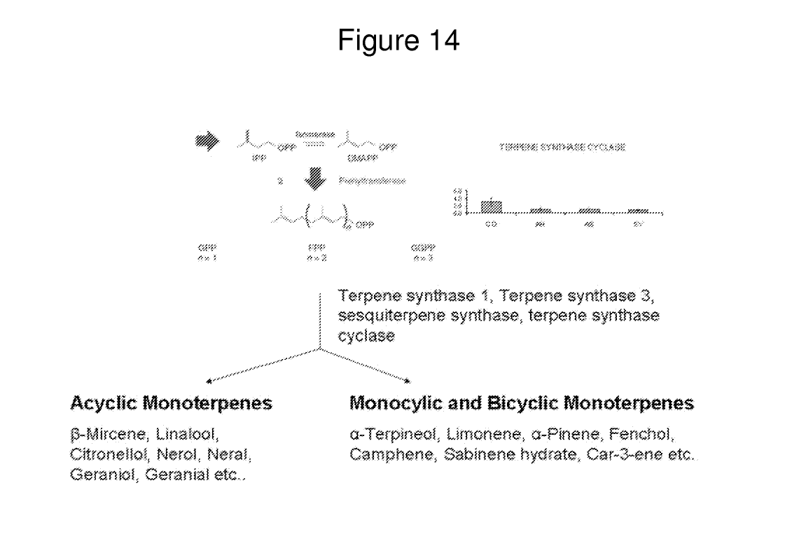

Genes encoding several types of terpene synthases involved in mono and diterpene biosynthesis were shown to be differentially regulated by HLB disease and this evidence might affect aroma composition and nutritional properties of citrus fruits (FIG. 12). These enzymes are involved in the synthesis and transport of a variety of terpenes, gibberellins, brassinoesteroids, alkaloids and plant volatiles, which play diverse roles in plant development and defense (Mercke et al., 2004). Two genes involved in terpenoid metabolism were analyzed using qRT-PCR in fruits and leaves of the four types of plants (FIGS. 12 and 13). Data confirmed the downregulation of terpenoid pathways in HLB-infected fruits. By contrast, in young leaves terpene synthase 3 and terpene synthase cyclase were induced by HLB disease while in mature leaves terpene synthase 3 was downregulated at both asymptomatic and symptomatic stage (FIG. 13). The transcriptional regulation of these terpenoid genes do not necessarily predict which particular type of terpene will be induced as our data cannot predict the substrate specificity of the encoded enzymes. What we can say is that the induction of these genes might induce differences in the production of acyclic, monocyclic and bicyclic monoterpenes (FIG. 14).

Another volatile pathway that appears to be induced is the salicylic acid-related pathway, as the induction of salicylic acid methyltransferase in response to HLB infection was observed in mature fruits and young leaves (FIG. 15). This gene is responsible of the conversion of salicylic acid in methylsalicylate and it is known to be induced after pathogen attacks in different plants (Loughrin et al., 1993; Huang et al., 2006). Gaseous MeSA produced in TMV-inoculated tobacco leaves acts as an airborne defense signal involved in the communication between infected and healthy plants and the amounts of gaseous MeSA produced after the infection were sufficient to induce expression of PR-1 proteins in nearby healthy tobacco plants (Shulaev et al., 1997). Although several pathogens induce the production of methylsalicylate, the identification of methylsalicylate in the volatile emissions from infected leaves and fruits may be used for HLB diagnosis.

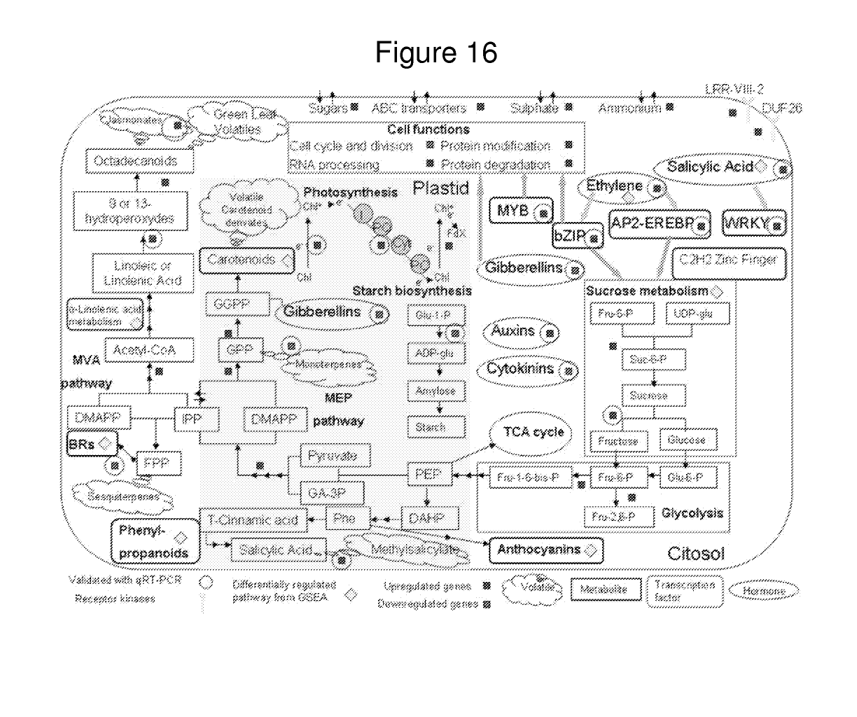

Apart from the pathways that lead to the production of volatile pathways we have identified many other biomarkers for early disease detection that are differentially regulated. We have constructed a network that connects different pathways based on known pathways and literature analysis. This network shown in FIG. 16 provides vivid insight into the metabolism in fruit induced by HLB disease. Carbohydrate metabolism is altered in the fruit with sucrose metabolism and glycolysis being severely affected, while in the plastid, several genes involved in light reactions were induced.

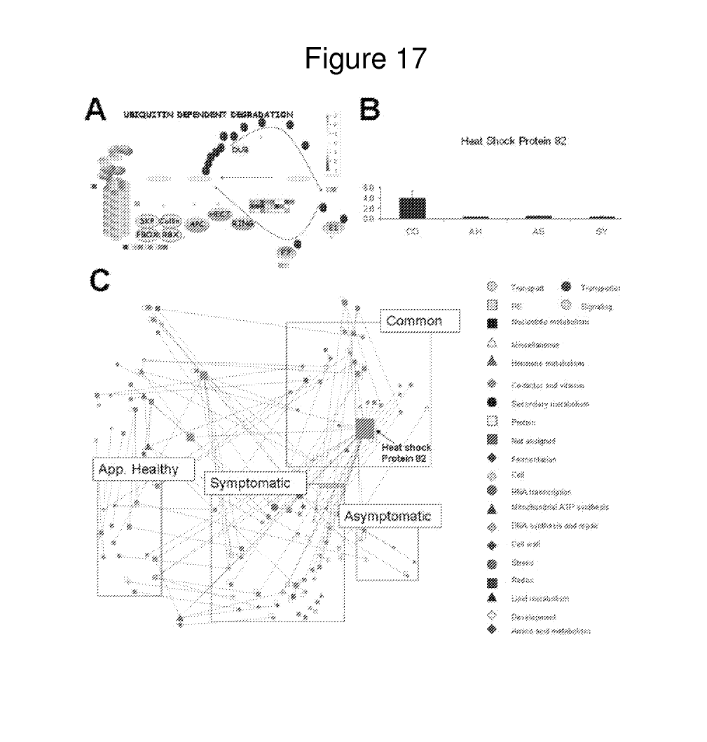

Hormone dysfunction may play a key role in the host response to HLB infection. It is interesting that gibberellin and cytokinin-related genes were mainly downregulated in symptomatic fruits while ethylene biosynthesis and signal transduction being induced. With respect to the regulation of cell functions, protein degradation and modification processes were highly affected by the disease. Indeed, genes involved in C3HC4-type RING finger proteins involved in ubiquitin degradation processes were differentially expressed in HLB-infected fruits (FIG. 17A). These results were linked with the down regulation of the gene heat shock protein 82 at both asymptomatic and symptomatic stage (FIG. 17B). This latter gene was identified by the construction of a biological regulatory network using literature-curated protein interaction datasets (Cusick et al., 2009) to deduce a predicted PPI network in citrus, visualized using graphviz software (FIG. 17C). Interestingly heat shock proteins (HSP82 and HSP70), highly interactive protein in the PPI network inferred in citrus, were downregulated in all three types of fruits from infected orchard (apparently healthy, asymptomatic and symptomatic stage). These proteins act as molecular chaperones to stabilize, reduce misfolding or facilitate refolding of proteins that have been denatured during stress events. In plant cells, HSP70 and HSP90 are involved in signal transduction leading to plant defenses. It is speculated that down regulation of heat shock proteins observed at different stage of HLB disease might enhance the protein misfolding processes in the fruit

### Example 6

### VOCs Associated with CTV

The effect of Twister-GC-TOF methodology on the CTV detection was examined. In this study, in addition to collecting healthy and CTV samples, we also collected samples from another category which was infected with two diseases called “CTV” and “stubborn”. In total, we obtained 12 CTV samples, 10 healthy samples and 11 CTV+stubborn samples.

The twister data set was generated as a peak table for univariate and multivariate data analysis. In total, 33 samples were analyzed and 383 common peaks were detected across the entire sample set; 125 BVOCs metabolites were identified with the remaining being 263 unidentified. The data was then subjected to principal component analysis (PCA) and partial least square discriminate analysis (PLS-DA) for classification and validation.

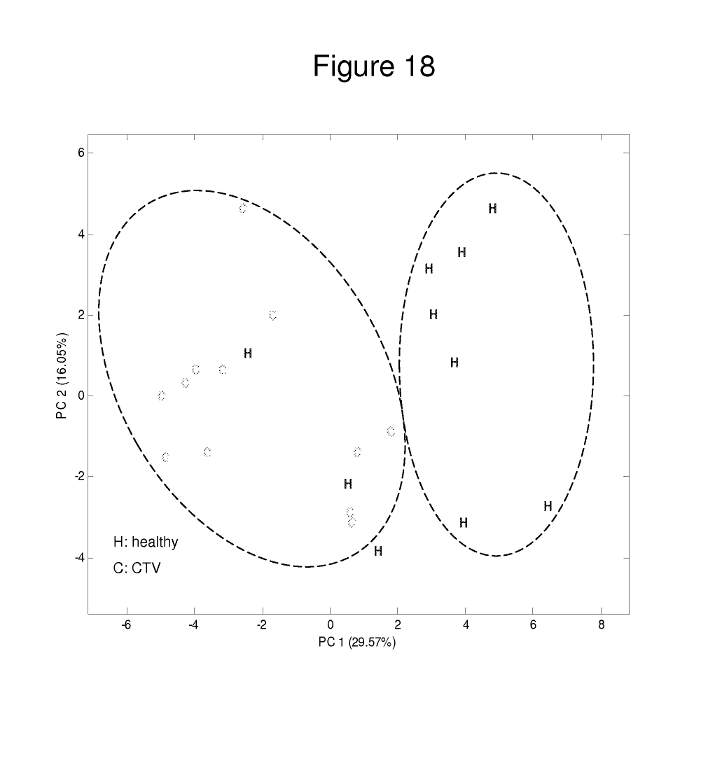

We have found the three categories of samples shared 383 peaks (120 of which have been identified). First, we applied Student's t-test to all the 383 peaks to have their p-values for a comparison between CTV and healthy. By setting up the p-value threshold to be <0.1, 41 peaks were retained for analysis. FIG. 18 shows a separation between CTV and healthy based on the 41 selected chemicals. Using leave-one-out validation strategy, PLSR yielded a classification accuracy of 86.36% (10/12 for CTV and 9/10 for control).

FIG. 18 is a PCA score plot of CTV vs. healthy only. Separation between the healthy and CTV infected can be observed, and with the exception of some possible outliers, the two classes can be broadly separated. Supervised analysis was then applied to the data set, and the accuracy of the model was then assessed using a leave-one-out validation (L-O-O) methodology. Using 4 PLS components, an accuracy of 86.36% was obtained. With loading plots and univariate analysis, VOC contributive toward the separation between healthy and CTV can be detected.

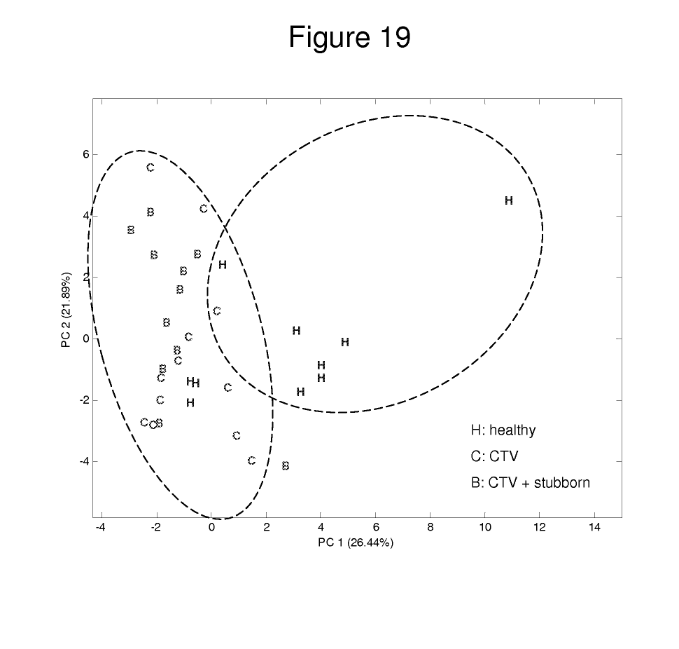

Then, we combined CTV and CTV+stubborn as a whole group and applied Student's t-test to have p-values for the comparison between healthy and the combined group. Also, using p<0.1 as a threshold value, 31 peaks were retained for analysis. FIG. 19 shows the separation between healthy and combined group based on the 31 peaks. Using leave-one-out validation strategy, PLSR yielded a classification accuracy of 84.85% (18/23 for combined group and 10/10 for control). This indicates a good detection of CTV infected trees, even if some were infected with another disease.

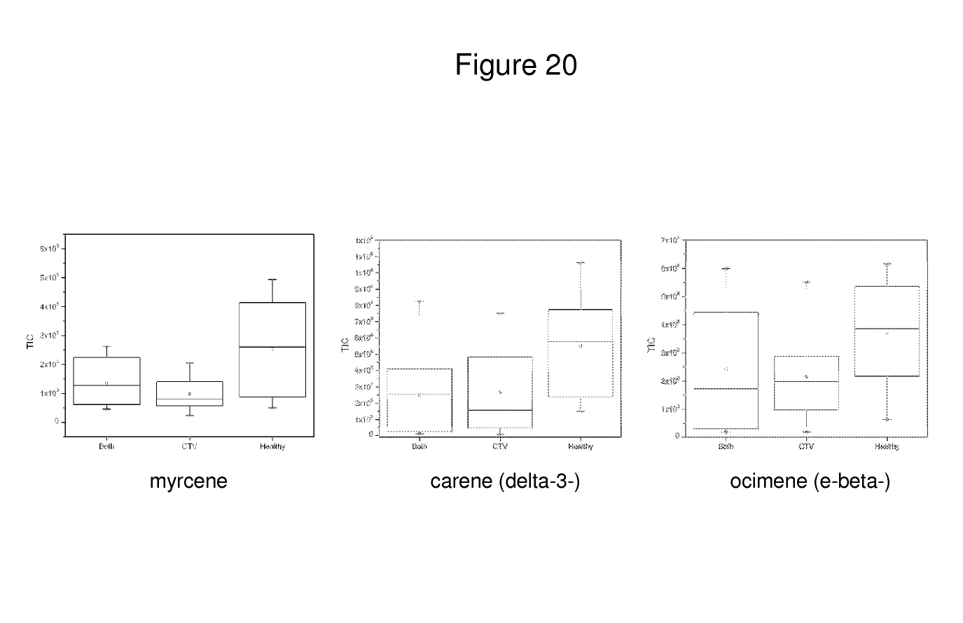

18 peaks are presented in both two separable peak sets (one for “Healthy versus CTV” and the other for “Healthy versus CTV relevant”), so we can assume they should have a more stable relationship with CTV. Three selected chemicals are myrcene, carene (delta-3-), and ocimene (e-beta-) (FIG. 20). Based on the 18 peaks, using the leave-one-out validation strategy, the accuracy on the “pure” CTV sample detection increased to 90.91% (10/12 for CTV and 10/10: for control),

Overall, the high diagnosis accuracies for various group comparisons demonstrated the effect of Twister-GC-TOF methodology on the CTV detection and CTV related biomarker detection.

### Example 7

### Comparison of VOCs from Valencia and Washington Navel

### Materials and Methods

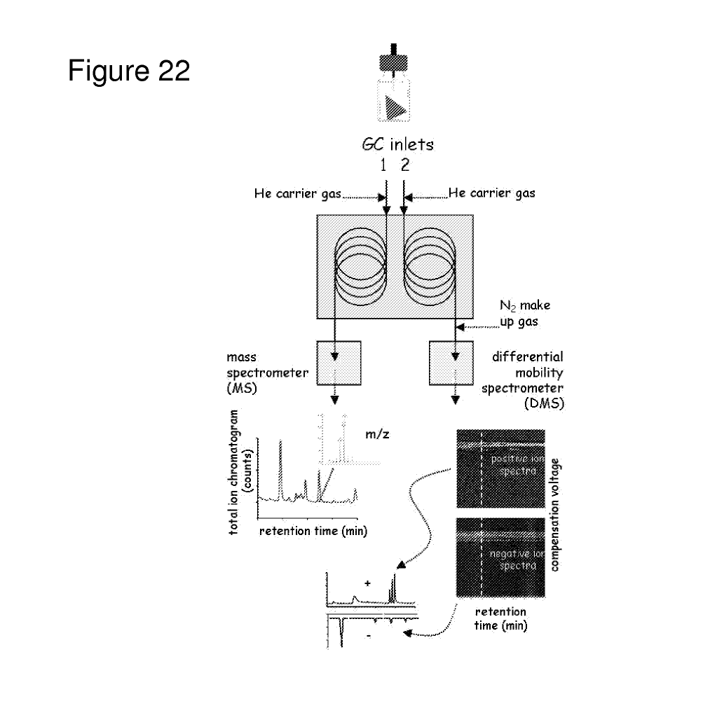

The VOC profiles emitted from citrus tree leaf samples were analyzed using hyphenated analytical instruments as shown in FIG. 22. A Varian Saturn 4000 series gas chromatograph electron ionization ion trap mass spectrometer (GC/EI-ITMS) (Varian; Walnut Creek, Calif.) was modified with the front two injection ports connected to two identical GC columns (VF-5 ms, Varian) residing in the same GC oven. Chemicals from two duplicate SPME fibers were desorbed simultaneously into the two injection ports, and the desorbed sample VOCs were subjected to the same GC oven temperature profile and then orthogonally detected with the two sensors. The eluting compounds from one column were analyzed using an ion trap mass spectrometer (left), while at the same time the other column output was connected to a DMS (right). The MS measurements allow for acquisition of mass-to-charge ratios (m/z) of fragment ionic species at specific retention times, which are unique for specific chemicals. By comparing these m/z traces to a standard NIST 08 and Wiley 09 databases, we can identify specific chemical compounds present within the VOC samples through MS matching. Likewise, the differential mobility spectrometer measures the positive and negative ion species abundances recorded as a function of sweeping compensation voltages.

### A. Citrus Samples for Experiments

Washington Navel and Valencia varieties of the dwarf citrus trees (Four Winds Growers, Inc.; Winters, Calif.) were purchased grafted onto the same rootstock, and were grown and stored in laboratory conditions under artificial white light and controlled temperature (21±2° C.). To measure the volatile profile of a leaf, we developed a method to capture these compounds. Individual leaves were separated from the tree above the rootstock graft, rinsed with deionized (DI) water, blotted dry, and immediately placed in 10 mL borosilicate glass headspace sampling vials and capped with a Teflon septa (Supelco; Walnut Creek, Calif.). The leaves were washed to remove any insecticide that covered the leaves: the presence of any insecticide would cause significant interference with both the GC/ITMS and GC/DMS spectra. Seventeen leaf samples from each varietal were collected in total (2 trees per varietal) for analysis.

The samples were placed in a temperature-controlled aluminum tray and maintained at 45° C. to equilibrate the volatile compounds emanating from the leaf into the headspace. Solid-phase microextraction (SPME, Supelco; Walnut Creek, Calif.) fibers coated with 85 μm polyacrylate were used to collect the volatiles. The fiber was inserted into the headspace and exposed for 1 hour. The sampling was carried out in pairs to allow for simultaneous GC/MS and GC/DMS analysis. Before each use, the SPME fibers were heated and conditioned at 250° C. for 90 min under constant flow of helium to remove any residual unrelated compounds adsorbed onto the polymer.

### B. Gas Chromatograph Mass Spectrometry (GC/MS)

To analyze chemical compounds adsorbed onto the SPME fiber, they were desorbed within the GC injection port for 7.5 min at 250° C. The chromatographic analysis was performed using a Varian Saturn 4000 series GC/ITMS, outfitted with a Combipal 3000 automated sampling handling system, using two 30 m×0.25 mm×0.25 μm phase columns (VF-5 ms, Varian) with stationary phase composition of 5% phenyl, 95% dimethylpolysiloxane. One of the analytical columns was connected to an ion trap mass spectrometer (Varian; Walnut Creek, Calif.), while the other identical column was connected to a differential mobility spectrometer (SVAC-1; Sionex; Bedford, Mass.). GC oven was cryochilled to 5° C. to help focus the initial desorbed volatiles onto the column heads prior to chromatographic separation. Both analytical columns were run with a flow rate of 1 mL/min of helium (Airgas, Inc.; Woodland, Calif.). The GC profiles were set as follows: initial temperature set at 5° C. hold for 15 min, ramp to 75° C. at 1° C./min with a hold of 15 min, ramp to 100° C. at 1° C./min and a hold of 15 min, ramp to 125° C. at 5° C./min with a hold of 5 min, ramp to 140° C. at 5° C./min. The injection port was maintained at 250° C. using a splitless injection to ensure complete transfer of all compounds into the analytical column.

The gas chromatograms were recorded using an ion trap mass spectrometer to produce a total ion chromatogram (TIC). The transfer line and the ion-trap manifold were maintained at 180° C. and 270° C., respectively. The analyte molecules were fragmented using an electron ionization source (70 eV). The MS scan range was set to record 35-400 Th range. The eluted chemical compounds were tentatively identified by comparing the ion fragmentation pattern with mass spectral database using NIST 08 and Wiley 09 mass spectral libraries using Mass Spectral search v2.0 software.

### C. Gas Chromatograph Differential Mobility Spectrometry (GC/DMS)

In the differential mobility spectrometry (DMS) unit used in this study, ions were generated from gas molecules when they travel past a sealed radioactive ⁶³Ni source, and indirectly through charge transfer between reactive ion carrier gas species that are also generated in this process. The ions then pass through electrodes with applied zero-average asymmetric radio frequency voltage pulses, with short strong positive pulses with long weak negative pulses.

The non-linearity in the ion mobility under week and high field conditions causes the ions to separate from one another, and produces two experimental recordings: one for the positively charged ion species, and the other for the negatively charged ion species. By applying a compensation voltage, selected ions are permitted to pass and their ion currents are registered. The signal amplitude reflects the chemical abundance in the sample. In this study, we used an RF of 1100V and a compensation voltage scan of −43 V to 15 V, and the grade 5 ultrapure nitrogen carrier was used at flow rate of 250 mL/min.

### GC/MS Data Analysis

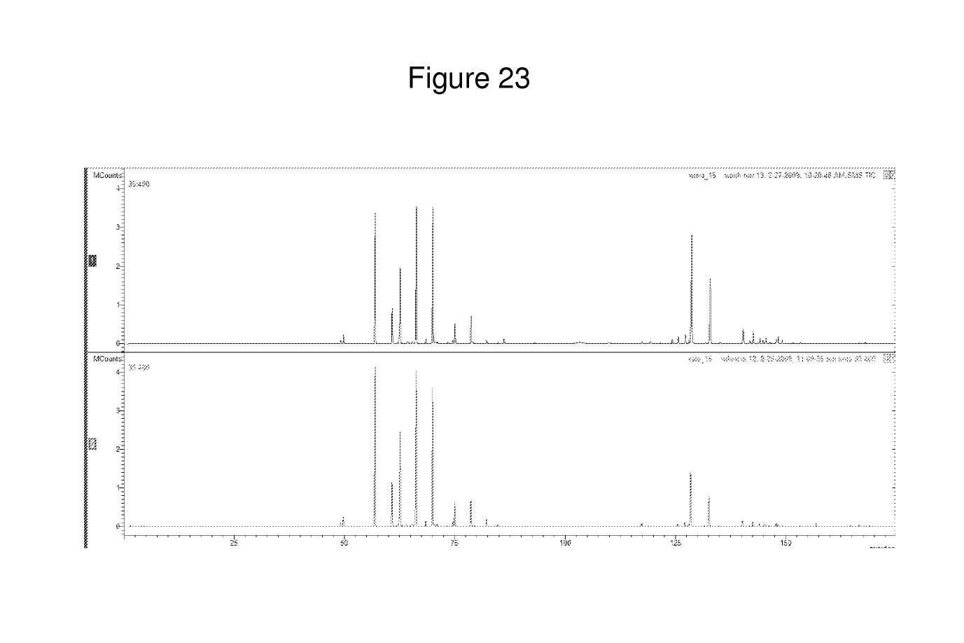

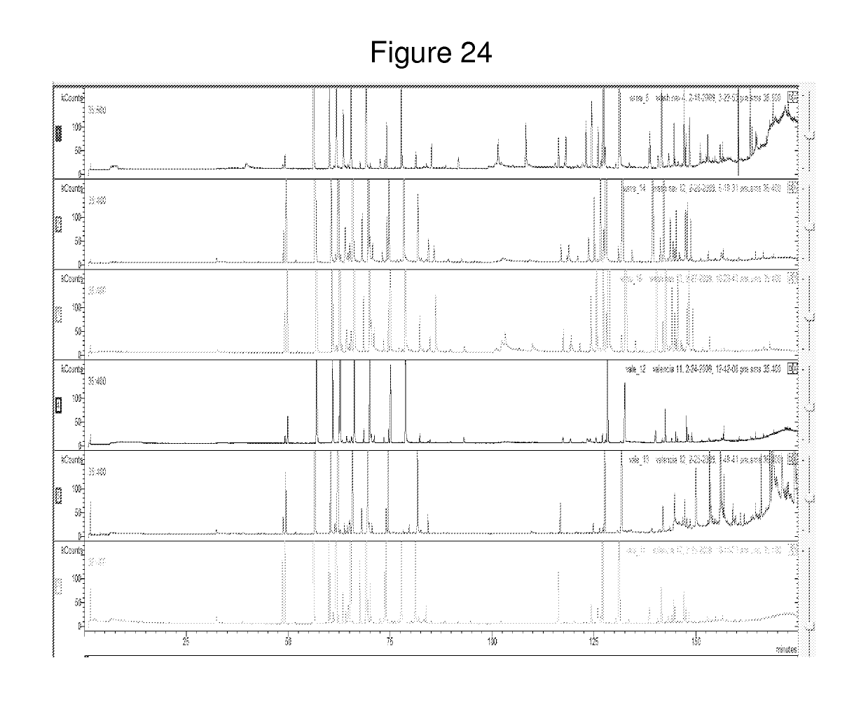

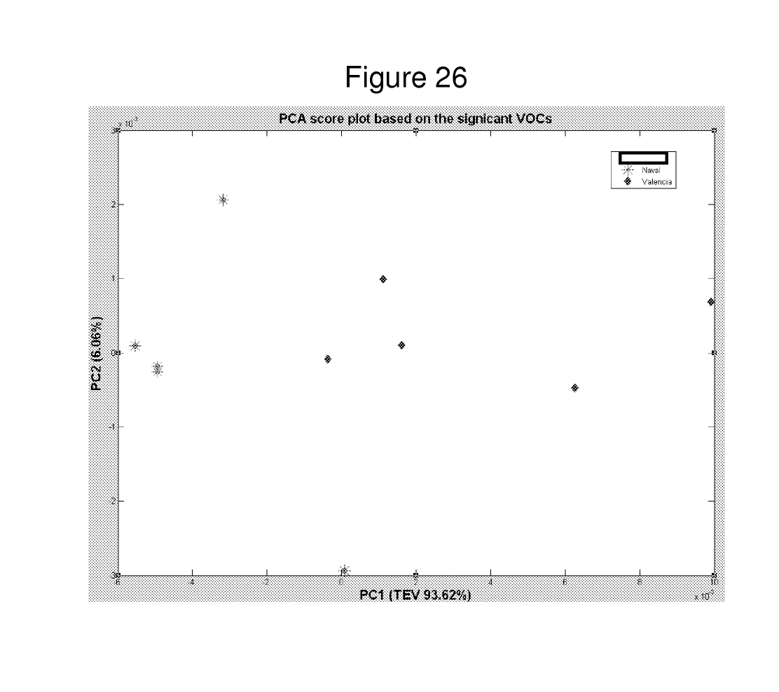

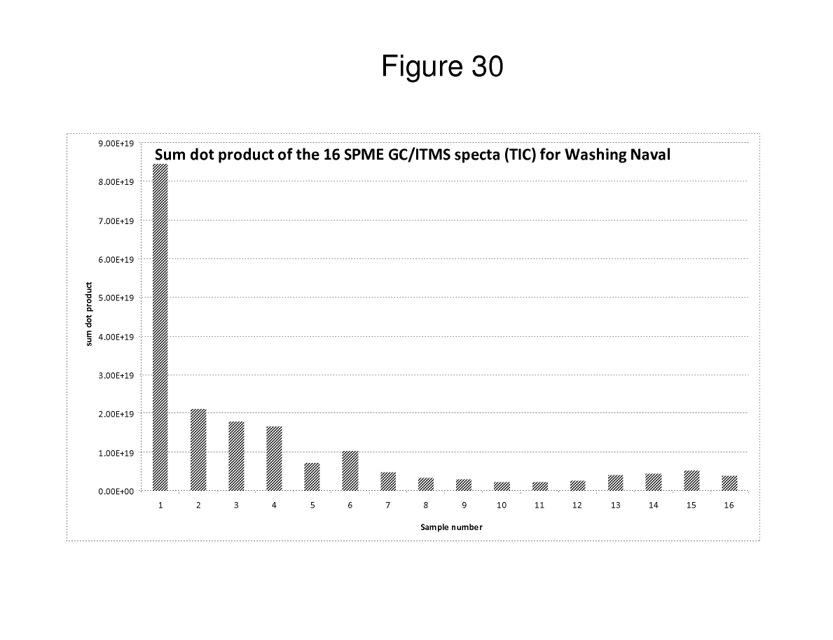

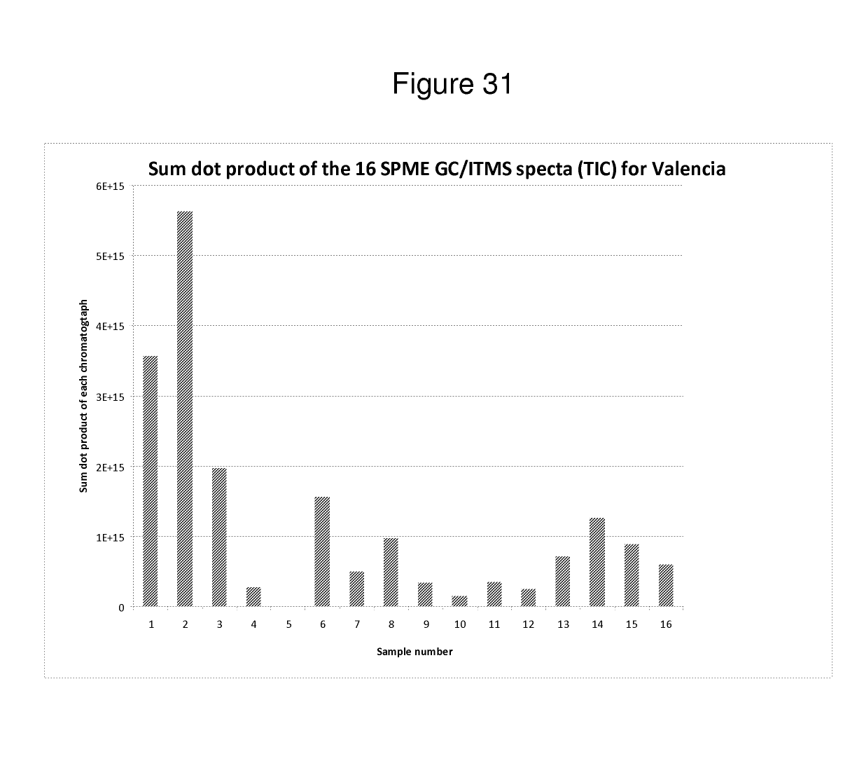

Seventeen samples from each citrus varietal were analyzed. The most reproducible chromatograms were chosen to form a balanced sample set for multivariate analysis. In total, 10 SPME GC/ITMS spectra were used, 5 from each varietal. They were chosen based on their sum dot product values, the lower the values, the more reproducible the results are (see FIGS. 30 and 31). Samples 11, 13, 14, 15, 16 were used (from both varietals). In preliminary studies, we observed variations in the abundance of VOCs proportional to the surface area of the leaf, which in turn varies with the size of the leaf tissue. Visual inspection of the SPME GC/ITMS data indicated there are two main prominent clusters of peaks ranging at 49-86 min and 119-149 min respectively, and the eluted peaks within the two respective clusters show highly conserved retention times and MS fragmentation profiles (FIG. 23 and Table 16). In FIG. 24, we display three GC profiles for Naval and Valencia; as can be seen, the VOC profiles are highly similar to each other. It is evident that there is a large degree of overlap between the VOCs produced by both varietals. Peak tables of all the major peaks within the TIC spectrum from both varietals were created via manual inspection and annotations, and retention time and fragmentation patterns were noted and tentatively matched against the NIST 08/Wiley 09 mass spectral databases using MS search V2.0 (see Table 16). The 41 VOC peaks were located; the VOC abundance was quantified by peak height; the subsequent peak tables were then subjected to multivariate analysis (PCA). In order to prevent any possible bias from confounding factors such as leaf size influencing classification, for each sample the height of a selected peak was normalized against the total abundance for all of the selected peaks (summed peak heights). Then, a Student's t-test was applied to detect statistically significant differing peaks. The separation of the two varietals based upon the discriminating peaks was then examined with PCA (FIG. 26).

### GC/DMS Data Analysis

The GC/DMS data are comprised of positive and negative ion spectra, each showing ion abundances as a function of retention time and compensation voltage. To examine the separation of the GC/DMS signals of the two varietals, we applied the Student's t-test to detect the differentiable pixels that had the potential to discriminate two varietals. To further examine the characteristics of the selected data points, PCA and principal component regression (PCR) were respectively employed to visually present the varietal differentiation based on these data points and quantitatively investigate the separability of the selected pixels.

### Results and Discussion

Given that plants are known to emanate a large number of volatile compounds, it is not surprising that we were able to detect a significant number of emitted VOCs through GC/MS and GC/DMS. Chromatographic peak analysis showed a wide range of both lower abundance compounds, as well as higher abundance more prevalent chemical species (FIG. 23). As we expected, we observed that many of the large abundance compounds overlap between the Washington Navel and Valencia citrus varietals, suggesting that the two may have very similar biochemical processes that results in the production of these emitted VOCs.

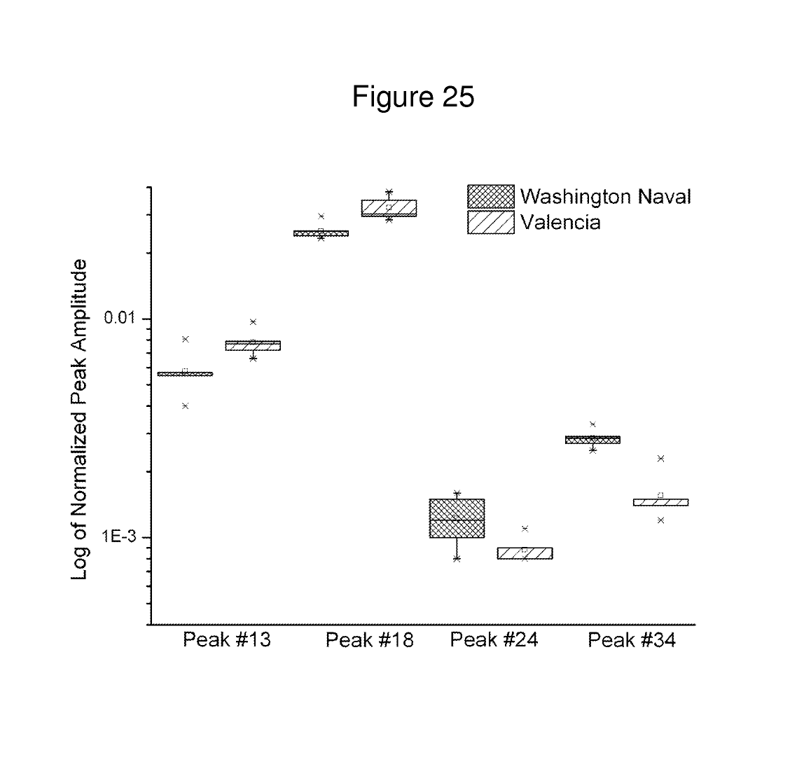

In total, 41 major VOCs were identified through manual inspection from the GC profiles in both Naval and Valencia varietals (Table 16). A Student t-test was then applied to the normalized peak table using a (p<0.05) threshold. Four VOCs that were found to be significantly different between the two varietals are listed in the Table 17 along with the possible chemical match(es). The normalized abundances (Y axis) of these four significant VOC peaks, showing differences in the VOCs abundances for the two varietals are shown on FIG. 25. The subsequent PCA analysis based on these four potentially discriminating VOCs was also performed (FIG. 26). The total variance captured by PC1 and PC2 was 93.62 and 6.06%, respectively, thus indicating that the majority of the variance within the data set is captured by PC1 with some minor contribution from PC2. Separation between the two groups is evident based on the 4 significant VOCs from our original Student's t-test.

Table 15 is a peak identification table of the 41 major VOCs prominent in both varietals, displaying the retention times, given peak ID, mass spectral profile and likely match within the NIST 05 database: there forward and reverse match score are as shown.

TABLE 16
Possible chemical match(es) for the GC peaks that were found to be
significantly different between the Navel and Valencia varietals.

| Peak | | |
|---|---|---|
| Number | R.T. (min) | Possible Chemical Match |
| 13 | 68.464 | 1R-α-Pinene |
| | | trans-β-Ocimene |
| | | cis-Ocimene |
| 18 | 75.037 | α-Humulene |
| | | 3-Carene |
| | | α-Terpinene |
| | | τ-Terpinene |
| 24 | 121.567 | Copaene |
| 34 | 141.903 | (+)-β-Funebrene |
| | | Cedrene |
| | | β-Sesquiphellandrene |
| | | Germacrene-D |

VOC production in plants changes in response to alterations in environmental conditions and reaches maxima at certain hours of the day when conditions (temperature, light intensity) are metabolically optimal for the plants to undergo photosynthesis and VOC production (Casado et al. 2008). By expanding the sampling to live leafs and to multiple times throughout the day, a more complete model relating to VOC production to natural day and night cycle could be obtained, rather than just a single snapshot in time. This could account for some of the variation observed in the volatile chemicals emitted from citrus in this study, and could also have large implications on exploiting appropriate VOC profile for non-invasive rapid disease diagnostics (Cevallos-Cevallos et al. 2009; Rouseff et al. 2008; Zhang and Hartung 2005).

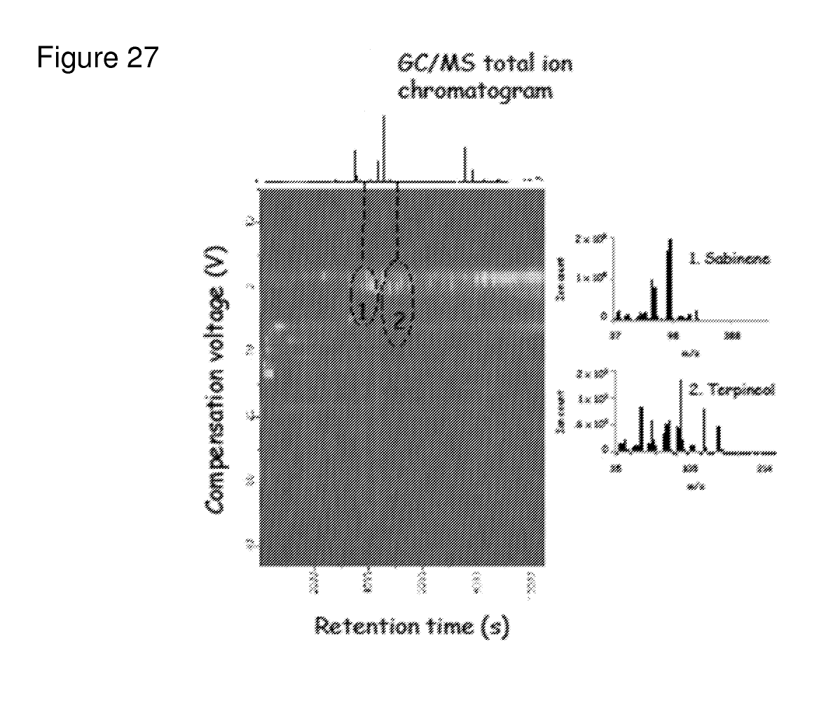

We profiled the VOC signature emitted from two citrus varietals using GC/DMS and GC/ITMS, as mentioned earlier. One of the advantages of an instrumentation setup with the front two injection ports connected identical analytical columns contained is that it allows simultaneous analysis of VOC samples to be run with parallel detection systems (FIG. 22). This dual system allowed us to build a library of chemical compound signatures for the GC/DMS that can be correlated to GC/ITMS data, meaning that it may be possible to determine chemical compound identification of peaks in both data sets. Two citrus leaf samples were prepared and two conditioned SPME fibers were used to sample the vial headspace above the leaves, (one SPME fiber per vial) and these were analyzed on the GC/ITMS and GC/DMS, respectively, as described in the Experimental section. The individual peaks on the GC/ITMS chromatogram were correlated with the output spectra of the DMS (FIG. 27). In this example, the DMS spectra are shown with two regions labeled #1-2, for which the aligned GC/ITMS mass spectra were used to identify the detected VOCs. The chemical matches were found to be compounds frequently observed in plant species: sabinene, carene, terpineol, and copaene. This figure conceptually illustrates how it is possible to move from identifying a peak in the GC/ITMS signal domain, to the matching equivalent peak in the GC/DMS domain. Such matching will allow us to establish a chemical library database for the DMS sensor. This also implies that it may be possible to locate important VOC metabolite biomarkers in the GC/DMS signal space that are not represented in the GC/ITMS data, or vice versa, due to differing sensitivities to certain chemicals of the two detectors.

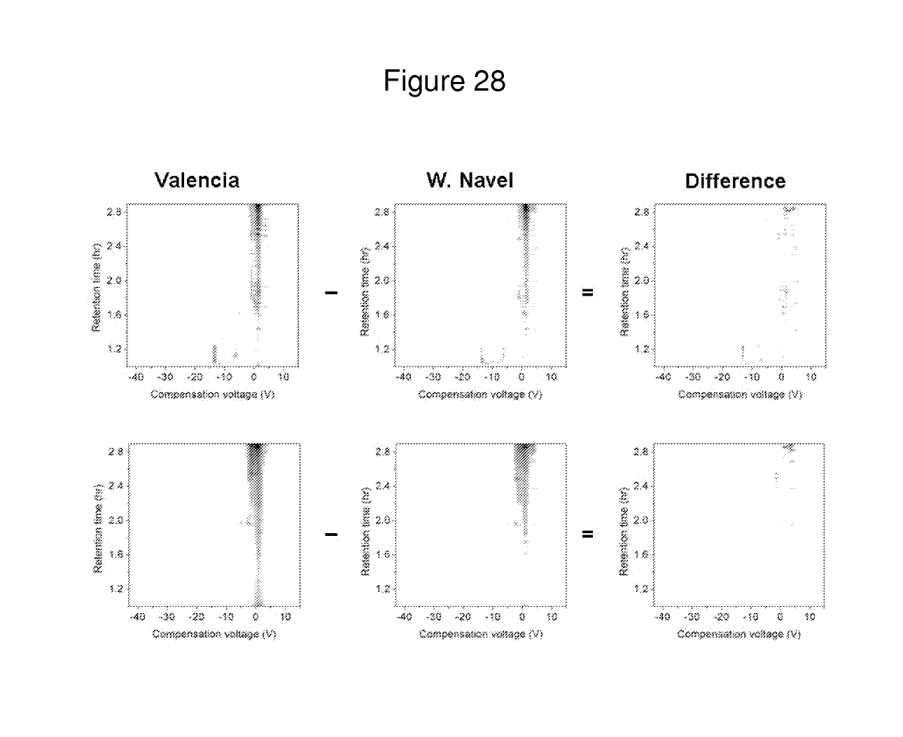

An intuitive method to examine the classification between the GC/DMS signals for the two varietals is to compare their mean signal intensities across the entire spectral plots (FIG. 28). In this case, we show the mean signal intensities for all points across both the positive and negative ion spectra of both varietals (n=13 for Washington Navel and n=14 for Valencia, left panels), and the signal-subtracted difference between the two varietal data sets (right panels). Both the positive and negative ion spectra show points within the spectra that are different between the Washington Navel and Valencia samples, suggesting that there may be some basis of separation of the VOC profiles generated for the different varietal groups in the GC/DMS biomarker space. In this case, if points within the spectra are highly variable for a single varietal data set, the averaged spectra would tend to minimize the importance of these points. Only the reproducible systematic point differences are notable within the subtracted images, and we find that there are several regions of these points within the data sets: more than were found within the traditional MS data sets.

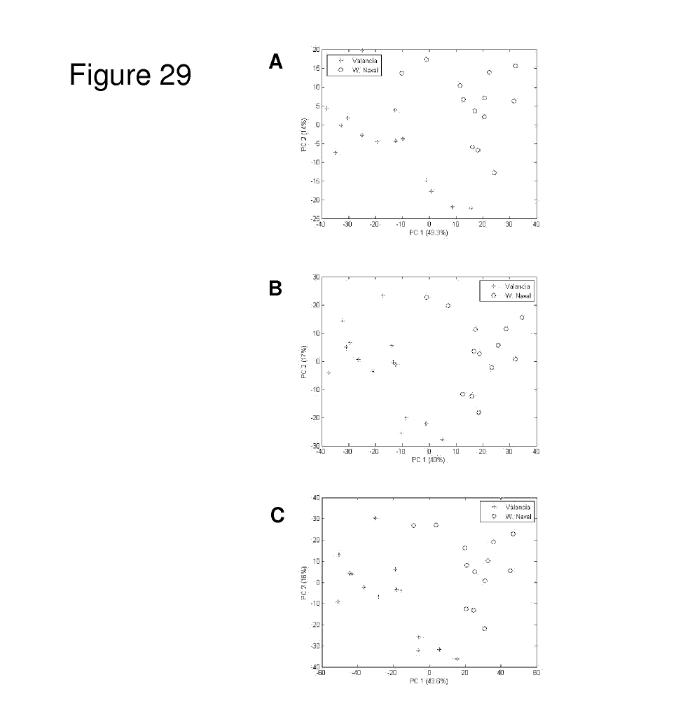

We applied the Student's t-test to detect the potential distinguishable features (pixels) between the varietals in the GC/DMS signal space (p<0.005), and detected 958 pixels within the positive signal and 1194 pixels within the negative signal that were statistically different between the two varietal groups. We then employed principal component analysis (PCA) to examine if these pixels contained sufficient information to separate the two varietals (FIG. 29). We found that the two varietals grouped well in both the positive ion spectra signal space (FIG. 29A), and the negative ion spectral signal space (FIG. 29B). When the two signal spaces were concatenated together and used simultaneously in the PCA, we found that the distinguishing power to separate the two groups was approximately equivalent (FIG. 29C).

Principal component regression (PCR) was then applied to quantitatively investigate the separability of these selected pixels. Similarly, the “leave-one-out” (LOO) strategy was employed for this task (Wold et al. 2001). The classification results are: 96% (PC number=7) based upon the positive spectra, 100% (PC number=3) on the negative spectra, and 100% (PC number=3) on the positive and negative integrated data.

Our studies indicate that although VOC differences can be measured between varietal citrus groups, these differences are relatively subtle. During field measurements, we expect there to be significant background sources of VOCs that may mask the chemical compounds of interest; thus, biochemical VOC libraries of citrus biomarkers are critical. We found that the GC/DMS was more effective at monitoring for many citrus volatiles than GC/ITMS alone, and this sensor may be a candidate platform to develop for in-field VOC monitoring in agriculture communities. Together, all of these results have strong implications for mobile VOC-detecting platforms developed for in-field diagnostic purposes, which is of particular interest to monitor the spread of vector-borne disease in citrus crops. Plant VOCs will vary depending upon the age of leaf and plant, temperature, season, stress condition, and other physiological and environmental factors. Analysis of the plant response during disease-caused stress will provide important clues for designing early disease detection to avoid the spread of disease. Biomarkers based upon VOCs will be a welcome departure from the state-of-the-art but slow biochemical assays that are currently used to track citrus diseases (Irey et al. 2006; Li et al. 2009; Li et al. 2008; Teixeira et al. 2005a; Teixeira et al. 2005b; Wang et al. 2006). For example, in citrus trees, traditional approaches of diagnosis of the devastating Huanglongbing disease rely on symptom appearance and pathogen detection in the field using real-time polymerase chain reaction technologies, which are problematic since the organism is not uniformly distributed within the tissues of infected trees and is therefore easily missed (Tatineni et al. 2008). The disease is asymptomatic during the primary spread of the pathogen through commercial orchards, and is associated with very low titers, undetectable with many current PCR methods (Teixeira et al. 2008). Disease-specific VOC biomarker changes may precede symptoms in plants allowing for potential asymptomatic detection. One important topic in future studies is to continue to characterize the VOCs associated with citrus disease that are present above the normal background VOC variations observed in these species. In those cases, the volatile biomarkers may point towards specific biochemical pathways within the plant tissue that are altered as the plant infection progresses, which will allow us to identify putative biochemical targets to slow or halt disease spread in this very important commodity crop. Even more important is the unambiguous chemical identification of the VOCs, many of which are present in low-abundances. Our results also highlight the need for developing correlated VOC libraries for in-field disease diagnosis using different sensor modalities as well as the development of higher order algorithms that enhance our understanding of the large data sets that these sensors will produce.

### Conclusions

We have developed a process to compare the expression and variability of volatile organic compounds from the leaves of Washington Navel and Valencia citrus plants. We found that SPME-based sample collection combined with both GC/ITMS and GC/DMS detection is relatively rapid, simple and reliable method for obtaining chemical information regarding the VOCs emitted in fresh leaves of citrus plants. We further demonstrated PCA as a promising method to examine the distribution of VOCs for different citrus varietals and PCR as a tool to quantitatively discriminate the varietals. Results obtained in this work suggest that the DMS data show a good potential to resolve additional low abundance chemicals compounds, complimenting the ITMS data. It is possible to correlate the data between the ITMS and DMS data set and identify the detected VOCs. Establishing VOC signatures can aid in the development of portable DMS sensor systems to detected volatiles in-field, and allow for real time analysis of plant health and distress response.

### Example 8

### Development of a Portable GC/DMS

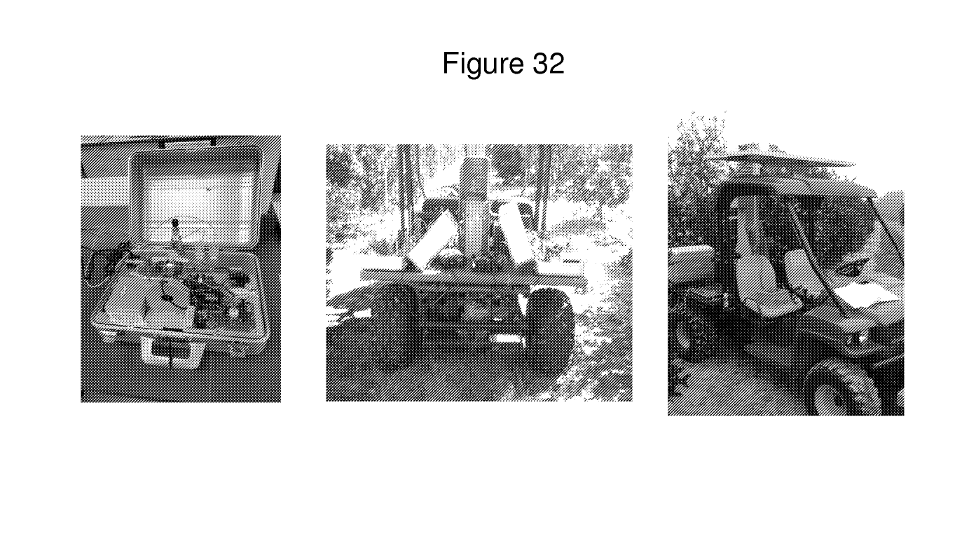

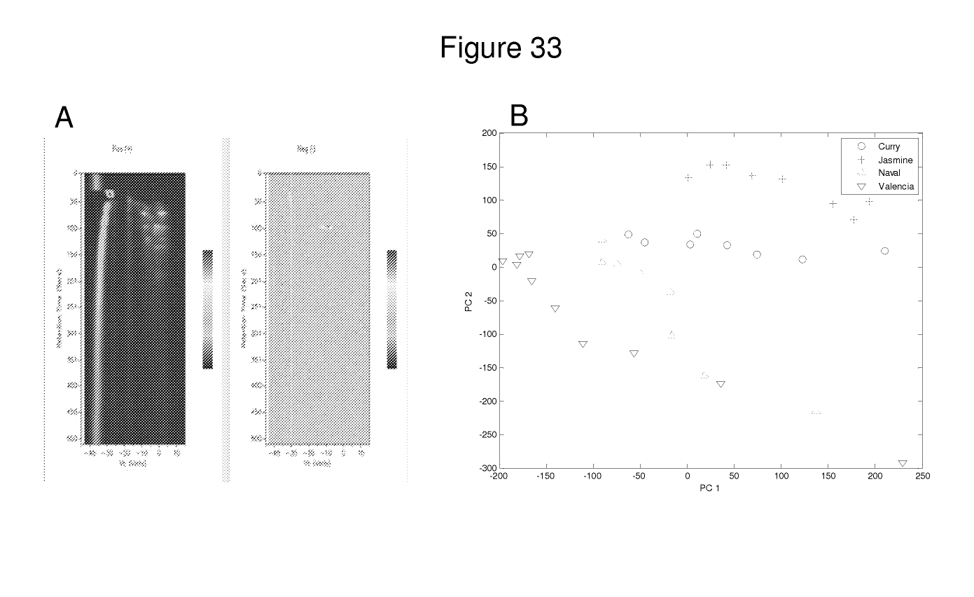

A portable DMS sensor system has been developed and applied to field sampling. FIG. 32 shows a typical situation of the field sampling process based on the portable DMS. To demonstrate the feasibility and reliability of the new system, we applied this new sensor system to a lab-scale benchmark experiment. Briefly, we collected and analyzed 10 samples for each of four kind of plants (Washington Naval, Orange jasmine, Indian curry, and Valencia) using this GC/DMS. The air containing VOCs to be analyzed was directly taken from leaf surface and each sample was just run for 10 minutes. The principal component distribution in FIG. 33 shows a clear separation of the four plant categories, which indicates the feasibility of using the GC/DMS “suitcase” for the VOC based citrus plant disease detection.

### Portable GC/DMS Based Citrus Disease Biomarker Detection

We are investigating VOC diagnostics for HLB detection. Prior to in-field sampling, we completed preliminary studies including sample collection protocol optimization from greenhouse citrus on the UC Davis campus. The in-field testing was carried out with collaborators at Lake Alfred, Fla. to account for weather and seasonal (trees blossoming, fruit harvest, etc.) variations. Trips were made in November 2010, December 2010, January 2011 and February-March 2011. This time frame included fruit ripening, fruit harvesting and blossoming. Sample collection was carried out in parallel using polymer-based absorption devices (SPME and Twister), as well as two portable GC/DMS analysis units. More than 100 samples were collected up to date using SPME, and analyzed using GC/MS. At least 200 individual GC/DMS runs were recorded for the same pair-matched trees used for the SPME sample collection. In addition, approximately 250 samples were taken using Twister devices (December 2010, February-March 2011). For all of the above experiments, trees of one varietal (Hamlin) were included. Samples were taken for HLB-infected and presumed healthy trees selected by scouts at the CREC facility in Lake Alfred Fla.

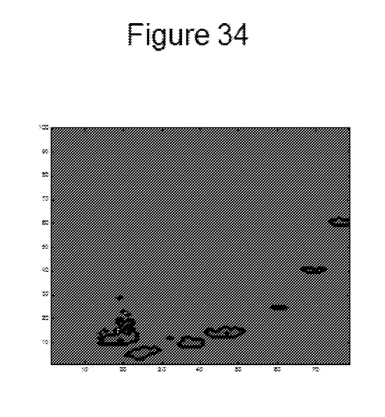

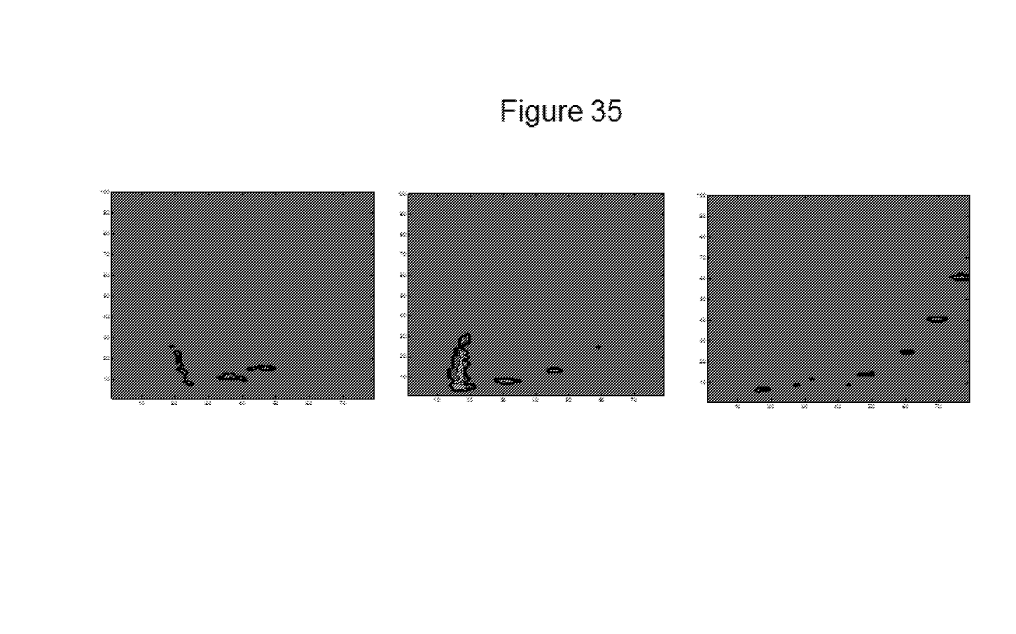

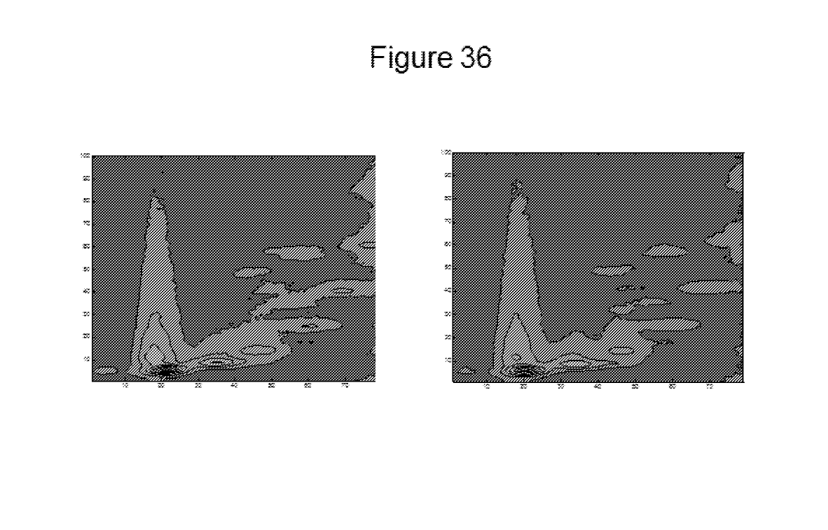

After comparing the stability of work conditions of the two GC/DMS “suitcases” in Florida, we chose to focus on the data from one “suitcase”. Samples collected in December and Jamnagar from this “suitcase”, were used for the biomarker detection work. In total, 55 healthy samples and 62 HLB samples were obtained in these two months. First, the Student's t-test, as a most widely used differentiation method, was applied to examine the separability of each pixel of the GC/DMS plot. FIG. 34 shows a Student's t-test p-value map, by setting up the p-value threshold to be 0.1. The brownish spots in this figure indicates potential biomarker areas, as they have both significantly low p-value (<0.1) and high enough signal intensity. In addition, we also applied principal component analysis to detect potential biomarkers, by examining the loading vector of a couple top principal components. FIG. 35 shows the areas which have larger loading coefficients (top 5%). As we know the larger the loading coefficients, the more contribution the corresponding pixels have. Therefore, the spots (from reddish to brownish) are the potential biomarker areas determined by principal component analysis. Clearly, FIGS. 34 and 35 have a big overlap of their selected spots, which suggests the reliability of the selected biomarker areas. To further examine the physical meaning of these selected potential biomarkers, we plotted the average spectra for both healthy and HLB samples in FIG. 36. It can be easily seen that the selected potential biomarker spots have a good agreement with the peak areas on the average GC/DMS spectra plots.

Then, we applied partial least square regression (PLSR) to the Student's t-test selected potential biomarker locations. Using leave-one-out strategy, we obtained a classification accuracy of 71% (PLS number=5). When we applied PLSR individually to the December samples and January samples, the accuracy was 80% for December and >95% for January. Both of them are higher than the accuracy of the combined sample set (i.e., 71%), suggesting a possible background variation between the two sampling times.

### Field and Greenhouse Testing of DMS Sensor Systems for Asymptomatic Disease Detection.

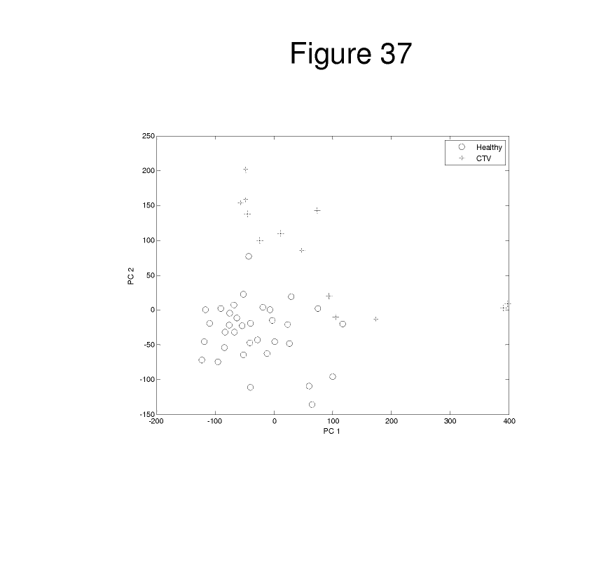

A large DMS dataset regarding CTV signature from severe, mildly infected citrus tree as well as from healthy controls (9 measurements per citrus tree), was collected, at present 180 DMS spectra. After excluding the severe noise contaminated samples or samples with unusual signal shifts, we have 13 CTV samples, 15 mild CTV samples, and 34 healthy samples. First PCA was applied to check the separation between healthy and CTV. It is clear from FIG. 37 that the first two principal components can already yield a good separation between two groups. Also, there is not a clear separation between the healthy samples collected from two different days, which may indicate a mild environmental variation. Using leave-one-out strategy, the accuracy based on PLSR model is 33/34 for healthy and 13/13 for HLB.

### Example 8

### GC/DMS Analysis of HLB Infection

GC/DMS data for HLB pathogen-affected citrus trees was collected using portable GC/DMS units. After sample screening, we retained 55 runs for healthy trees and 62 for HLB-affected trees from a whole sample set collected with a GC/DMS.

We applied the Student's t-test to each pixel to examine its separability between HLB and healthy. We plotted a p-value map. The pixels with p-value (<0.1) are uniformly colored with “red” and the other pixels with “blue”. These “red” pixels are potential biomarkers. Comparing the average spectral plot for Healthy and HLB, we can further confirm the physical meaning of these “red” pixels (p<0.1).

Using the selected pixels (p<0.1), PLSR model with cross-validating strategy yielded a HLB detection accuracy of 71%.

### Wavelet Transformation Provides a Useful Tool for Data Dimension Reduction and Signal Concentrating (Feature Extraction)

With wavelet transformation, raw spectral data can be decomposed into a low frequency part and a high frequency part. Low frequency part, which usually corresponds to real signal, can be further decomposed into next level. According to our experience (Zhao et al., 2009; Felinger and Kare, 2004), we decomposed the raw data into the 3rd level. In this study, the low frequency coefficients at the 3rd level were used as a representation of raw data for detection analysis.

### Wavelet Transformation Helps Increase Accuracy and the First 3 Minutes Signal Yields an Even Higher Accuracy.

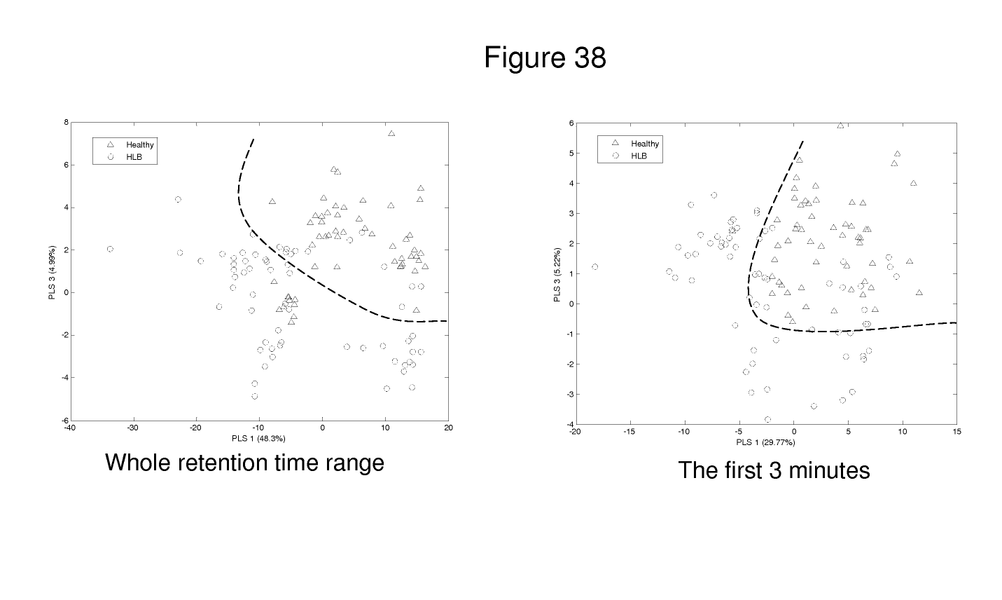

For each sample spectral data, we respectively applied wavelet transformation to the whole retention time range and the first 3 minutes (FIG. 38). Then PLSR was applied to the wavelet coefficients to quantitatively validate the classification accuracy.

Using cross-validation strategy, PLSR yielded an accuracy of 78% for the classification based on the whole time range and an accuracy of 82% for that based on the first 3 minutes. Both of these are higher than the accuracy based on the t-test selected pixels, and the first 3 minutes seem to have an equally good or even better detection result than the whole time range.

### Example 9

### GC/DMS Analysis of CTV Infection

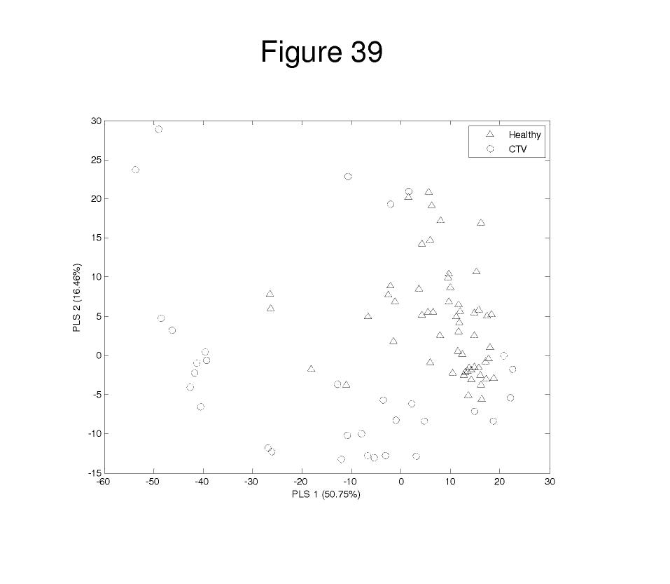

GC/DMS data for CTV pathogen-affected citrus trees was collected using portable GC/DMS units. After screening a sample collected from Pauma Valley, Calif., we retained 58 runs for Healthy trees and 51 runs for CTV-affected trees. Wavelet analysis was applied to each sample spectrum and PLSR was used to quantitatively validate the detection accuracy. A 3-level wavelet transformation was applied to each sample spectrum. Using leave-one-out validation strategy, a detection accuracy of 97.25% was obtained (56/58 for healthy and 50/51 for CTV) (FIG. 39).

REFERENCES

| No. | Reference |
|---|---|
| 1. | Agelopoulos, N. G., et al., A Novel Approach for Isolation of Volatile Chemicals Released by |
| | Individual Leaves of a Plant in situ. Journal of Chemical Ecology, 1999. 25(6): p. 1411-1425. |
| 2. | Avijit Roy, R. H. Brlansky (2010) Genome analysis of an orange stem pitting citrus tristeza |
| | virus isolate reveals a novel recombinant genotype. Virus Research 151 (2010) 118-130 |
| 3. | Ayer S, Zhao W, Davis CE (2008) Differentiation of proteins and viruses using pyrolysis gas |
| | chromatography differential mobility spectrometry (Py/GC/DMS) and pattern recognition. |
| | IEEE Sensors Journal 8: 1586-1592 |
| 4. | Baldwin IT, Halitschke R, Paschold A, von Dahl CC, Preston CA (2006) Volatile Signaling in |
| | Plant-Plant Interactions: “Talking Trees” in the Genomics Era. Science 311: 812-815 |
| 5. | Bar-Joseph, M., S. M. Garnsey, D. Gonsalves, M. Moscovitz, D. E. Purcifull, M. F. Clark, |
| | and G. Loebenstein (1979). The use of enzyme-linked immunosorbent assay for detection of |
| | citrus tristez virus. Phytopathology 69: 190-194. |
| 6. | Bezemer TM, van Dam NM (2005) Linking aboveground and belowground interactions via |
| | induced plant defenses. Trends in Ecology & Evolution 20: 617-624 |
| 7. | Bové J. M. 2006. Huanglongbing: A destructive, newly-emerging, century old disease of |
| | citrus. J. Plant Pathology, 88: 7-37. |
| 8. | Bullinger, D., et al., Bioinformatical evaluation of modified nucleosides as biomedical |
| | markers in diagnosis of breast cancer. Analytica Chimica Acta, 2008. 618(1): p. 29-34. |
| 9. | Carter D. J., Cary R. B. 2007. Lateral flow microarrays: A novel platform for rapid nucleic acid |
| | detection based on miniaturized lateral flow chromatography. Nucleic Acids Research, 35 |
| | (10), art. no. e74. |
| 10. | Cary R. B. 2007. Miniaturized lateral flow device for rapid and sensitive detection of proteins |
| | or nucleic acids. U.S.P.T.O. |
| 11. | Casado D, Gemeno C, Avilla J, Riba M (2008) Diurnal variation of walnut tree volatiles and |
| | electrophysiological responses in Cydia pomonella (Lepidoptera: Tortricidae). Pest |
| | Management Science 64: 736-747 |
| 12. | Cevallos-Cevallos JM, Rouseff R, Reyes-De-Corcuera J (2009) Untargeted metabolite |
| | analysis of healthy and Huanglongbing-infected orange leaves by CE-DAD. Electrophoresis |
| | 30: 1240-1247 |
| 13. | Cusick ME, Yu HY, Smolyar A, Venkatesan K, Carvunis AR, Simonis N, Rual JF, Borick H, |
| | Braun P, Dreze M, Vandenhaute J, Galli M, Yazaki J, Hill DE, Ecker JR, Roth FP, Vidal M. |
| | 2009. Literature-curated protein interaction datasets. Nature methods, 6(1): 39-46. |
| 14. | Dafoe NJ, Zamani A, Ekramoddoullah AKM, Lippert D, Bohlmann J, Constabel CP (2009) |
| | Analysis of the poplar phloem proteome and its response to leaf wounding. Journal of |
| | Proteome Research 8: 2341-2350 |
| 15. | do Carmo Teixeira D., Saillard C., Eveillard S., Danet J. L., da Costa P. I., Ayres A. J., Bove J. |
| | 2005. Candidatus Liberibacter americanus', associated with citrus huanglongbing (greening |
| | disease) in Sao Paulo State, Brazil. International Journal of Systematic and Evolutionary |
| | Microbiology, 55 (5): 1857-1862. |
| 16. | Donaire L, Wang Y, Gonzalez-Ibeas D, Mayer KF, Aranda MA, et al. (2009) Deep- |
| | sequencing of plant viral small RNAs reveals effective and widespread targeting of viral |
| | genomes. Virology 392: 203-214. |
| 17. | Dudareva N, Pichersky E, Gershenzon J (2004) Biochemistry of Plant Volatiles. Plant |
| | Physiol. 135: 1893-1902 |
| 18. | Felinger, A. and Kare (2004) Wavelet analysis of the baselind noise in HPLC. Chemometrics |
| | and Intelligent laboratory Systems 72(2): 225-232. |
| 19. | Freitas, M. P., Multivariate image analysis applied to QSAR: Evaluation to a series of |
| | potential anxiolytic agents. Chemometrics and Intelligent Laboratory Systems, 2008. 91(2): p. |
| | 173-176. |
| 20. | Friederike Bruessow a, Mar\acute{ }a J. Asins b, Josep A. Jacas c, Alberto Urbaneja. (2010) |
| | Replacement of CTV-susceptible sour orange rootstock by CTV-tolerant ones may have |
| | triggered outbreaks of Tetranychus urticae in Spanish citrus. Agriculture, Ecosystems and |
| | Environment 137 (2010) 93-98. |
| 21. | Glendinning J, Foley C, Loncar I, Rai M (2009) Induced preference for host plant chemicals |
| | in the tobacco hornworm: contribution of olfaction and taste. Journal of Comparative |
| | Physiology. A, Neuroethology, Sensory, Neural, and Behavioral Physiology |
| 22. | Goff SA, Klee HJ (2006) Plant Volatile Compounds: Sensory Cues for Health and Nutritional |
| | Value? Science 311: 815-819 |
| 23. | Herrmann U, Jonischkeit T, Bargon J, Hahn U, Li Q-Y, Schalley CA, Vogel E, Vögtle F |
| | (2002) Monitoring apple flavor by use of quartz microbalances. Analytical and Bioanalytical |
| | Chemistry 372: 611-614 |
| 24. | Hocquellet A., Toorawa P., Bove J. M., Garnier M. 1999. Detection and identification of the |
| | two Candidatus Liberobacter species associated with citrus huanglongbing by PCR |
| | amplification of ribosomal protein genes of the beta operon. Molecular and cellular probes, 13 |
| | (5): 373-379. |
| 25. | Huang WE, Huang LF, Preston GM, Naylor M, Carr JP, Li YH, Singer AC, Whiteley AS, |
| | Wang H. 2006. Quantitative in situ assay of salicylic acid in tobacco leaves using a genetically |
| | modified biosensor strain of Acinetobacter sp ADP1. Plant Journal, 46 (6): 1073-1083. |
| 26. | Irey MS, Gast T, Gottwald TR (2006) Comparison of visual assessment and polymerase chain |
| | assay testing to estimate the incidence of the Huanglongbing pathogen in commercial Florida |
| | citrus. Proceedings of the 119th Annual Meeting of the Florida State Horticultural Society |
| | 119: 89-93 |
| 27. | Jagoueix S, Bove J. M., Garnier M. 1996. PCR detection of the two ‘Candidatus’ liberobacter |
| | species associated with greening disease of citrus. Molecular and cellular probes, 10 (1): 43-50. |
| 28. | Jagoueix S., Bové J. M., Garnier M. 1994. The Phloem-Limited Bacterium of Greening |
| | Disease of Citrus is a Member of the alpha Subdivision of the Proteobacteria. Int. J. System. |
| | Bacteriology, 44: 379-386. |
| 29. | Karl T, Guenther A, Turnipseed A, Patton EG, Jardine K (2008) Chemical sensing of plant |
| | stress at the ecosystem scale. Biogeosciences Discuss. 5: 2381-2399 |
| 30. | Kessler A, Baldwin IT (2001) Defensive Function of Herbivore-Induced Plant Volatile |
| | Emissions in Nature. Science 291: 2141-2144 |
| 31. | Kishimoto K, Matsui K, Ozawa R, Takabayashi J (2005) Volatile C6-aldehydes and allo- |
| | ocimene activate defense genes and induce resistance against Botrytis cinerea in Arabidopsis |
| | thaliana. Plant and Cell Physiology 46: 1093-1102 |
| 32. | Lee J-E, Hong Y-S, Lee C-H (2009) Characterization of fermentative behaviors of lactic acid |
| | bacteria in grape wines through 1H NMR- and GC-based metabolic profiling. Journal of |
| | Agricultural and Food Chemistry 57: 4810-4817 |
| 33. | Li W., Hartung J. S., Levy L. E. 2006. Quantitative real time PCR for detection and |
| | identification of Candidatus Liberibacter species associated with citrus huanglongbing. J. |
| | Microbiological Methods, 66: 104-115. |
| 34. | Li WB, Levy L, Hartung JS (2009) Quantitative Distribution of ‘Candidatus Liberibacter |
| | asiaticus’ in Citrus Plants with Citrus Huanglongbing. Phytopathology 99(2): 139-144 |
| 35. | Li WB, Li DY, Twieg E, Hartung JS, Levy L (2008) Optimized quantification of unculturable |
| | Candidatus Liberibacter spp. in host plants using real-time PCR. Plant Disease 92: 854-861 |
| 36. | Loreto F, Barta C, Brilli F, Nogues I (2006) On the induction of volatile organic compound |
| | emissions by plants as consequence of wounding or fluctuations of light and temperature. |
| | Plant, Cell & Environment 29: 1820-1828 |
| 37. | Loughrin JH, Hamiltonkemp TR, Burton HR, Andersen RA. 1993. Effect of diurnal sampling |
| | on the headspace of detached nicottiana-suaveolens flowers. 1993. Phytochemistry, 32 (6): |
| | 1417-1419. |
| 38. | Lu Y, Harrington PB (2007) Forensic application of gas chromatography - Differential |
| | mobility spectrometry with two-way classification of ignitable liquids from fire debris. |
| | Analytical Chemistry 79: 6752-6759 |
| 39. | Lytovchenko A, Beleggia R, Schauer N, Isaacson T, Leuendorf J, Hellmann H, Rose J, Fernie A |
| | (2009) Application of GC-MS for the detection of lipophilic compounds in diverse plant |
| | tissues. Plant Methods 5: 4-4 |
| 40. | Mercke P, Kappers IF, Verstappen FWA, Vorst O, Dicke M, Bouwmeester HJ. 2004. |
| | Combined transcript and metabolite analysis reveals genes involved in spider mite induced |
| | volatile formation in cucumber plants. Plant Physiology, 135 (4): 2012-2024. |
| 41. | Müller K, Pelzing M, Gnauk T, Kappe A, Teichmann U, Spindler G, Haferkorn S, Jahn Y, |
| | Herrmann H (2002) Monoterpene emissions and carbonyl compound air concentrations |
| | during the blooming period of rape (Brassica napus). Chemosphere 49: 1247-1256 |
| 42. | Navarro B, Pantaleo V, Gisel A, Moxon S, Dalmay T, et al. (2009) Deep Sequencing of |
| | Viroid-Derived Small RNAs from Grapevine Provides New Insights on the Role of RNA |
| | Silencing in Plant-Viroid Interaction. Plos One 4. |
| 43. | Paolini J, Tomi P, Bernardini A-F, Bradesi P, Casanova J, Kaloustian J (2008) Detailed |
| | analysis of the essential oil from Cistus albidus L. by combination of GC/RI, GC/MS and |
| | 13C-NMR spectroscopy. Natural Product Research 22: 1276-1284 |
| 44. | Park SW, Kaimoyo E, Kumar D, Mosher S, Klessig DF (2007) Methyl salicylate is a critical |
| | mobile signal for plant systemic acquired resistance. Science 318: 113-116 |
| 45. | Prasad S, Pierce KM, Schmidt H, Rao JV, Guth R, Bader S, Synovec RE, Smith GB, Eiceman GA |
| | (2007) Analysis of bacteria by pyrolysis gas chromatography-differential mobility |
| | spectrometry and isolation of chemical components with a dependence on growth |
| | temperature. Analyst 132: 1031-1039 |
| 46. | Prestes RA, Colnago LA, Forato LA, Carrilho E, Bassanezi RB, Wulff NA (2009) Nuclear |
| | magnetic resonance characterization of metabolite disorder in orange trees caused by citrus |
| | sudden death disease. Molecular Plant Pathology 10: 51-57 |
| 47. | Rapparini F, Baraldi R, Facini O (2001) Seasonal variation of monoterpene emission from |
| | Malus domestica and Prunus avium. Phytochemistry 57: 681-687 |
| 48. | Rearden P, Harrington PB, Karnes JJ, Bunker CE (2007) Fuzzy rule-building expert system |
| | classification of fuel using solid-phase microextraction two-way gas chromatography |
| | differential mobility spectrometric data. Analytical Chemistry 79: 1485-1491 |
| 49. | Rohloff J, Bones AM (2005) Volatile profiling of Arabidopsis thaliana - Putative olfactory |
| | compounds in plant communication. Phytochemistry 66: 1941-1955 |
| 50. | Rouseff RL, Onagbola EO, Smoot JM, Stelinski LL (2008) Sulfur volatiles in guava (Psidium |
| | guajava L.) leaves: Possible defense mechanism. Journal of Agricultural and Food Chemistry |
| | 56: 8905-8910 |
| 51. | Runyon JB, Mescher MC, De Moraes CM (2006) Volatile Chemical Cues Guide Host |
| | Location and Host Selection by Parasitic Plants. Science 313: 1964-1967 |
| 52. | Schmidt H, Tadjimukhamedov F, Mohrenz IV, Smith GB, Eiceman GA (2004) |
| | Microfabricated differential mobility spectrometry with pyrolysis gas chromatography for |
| | chemical characterization of bacteria. Analytical Chemistry 76: 5208-5217 |
| 53. | Shnayderman M, Mansfield B, Yip P, Clark HA, Krebs MD, Cohen SJ, Zeskind JE, Ryan ET, |
| | Dorkin HL, Callahan MV, Stair TO, Gelfand JA, Gill CJ, Hitt B, Davis CE (2005) Species- |
| | specific bacteria identification using differential mobility spectrometry and bioinformatics |
| | pattern recognition. Analytical Chemistry 77: 5930-5937 |
| 54. | Shulaev V, Silverman P, Raskin I. 1997. Airborne signalling by methyl salicylate in plant |
| | pathogen resistance. Nature, 385 (6618): 718-721. |
| 55. | Stewart-Jones A, Poppy G (2006) Comparison of Glass Vessels and Plastic Bags for |
| | Enclosing Living Plant Parts for Headspace Analysis. Journal of Chemical Ecology 32: 845-864 |
| 56. | Sumner LW, Mendes P, Dixon RA (2003) Plant metabolomics: large-scale phytochemistry in |
| | the functional genomics era. Phytochemistry 62: 817-836 |
| 57. | Tatineni S, Sagaram US, Gowda S, Robertson CJ, Dawson WO, Iwanami T, Wang N (2008) |
| | In planta distribution of ‘Candidatus Liberibacter asiaticus’ as revealed by polymerase chain |
| | reaction (PCR) and real-time PCR. Phytopathology 98(5): 592-599 |
| 58. | Teixeira DC, Saillard C, Couture C, Martins EC, Wulff NA, Eveillard-Jagoueix S, Yamamoto PT, |
| | Ayres AJ, Bove JM (2008) Distribution and quantification of Candidatus Liberibacter |
| | americanus, agent of huanglongbing disease of citrus in Sao Paulo State, Brasil, in leaves of |
| | an affected sweet orange tree as determined by PCR. Molecular and Cellular Probes 22: 139-150 |
| 59. | Teixeira DD, Danet JL, Eveillard S, Martins EC, Junior WCJ, Yamamoto PT, Lopes SA, |
| | Bassanezi RB, Ayres AJ, Saillard C, Bove JM (2005a) Citrus huanglongbing in Sao Paulo |
| | State, Brazil: PCR detection of the ‘Candidatus’ Liberibacter species associated with the |
| | disease. Molecular and Cellular Probes 19: 173-179 |
| 60. | Teixeira DD, Saillard C, Eveillard S, Danet JL, da Costa PI, Ayres AJ, Bove J (2005b) |
| | ‘Candidatus Liberibacter americanus’, associated with citrus huanglongbing (greening disease) |
| | in Sao Paulo State, Brazil. International Journal of Systematic and Evolutionary Microbiology |
| | 55: 1857-1862 |
| 61. | Tholl D, Boland W, Hansel A, Loreto F, Roese USR, Schnitzler Jr-P (2006) Practical |
| | approaches to plant volatile analysis. The Plant Journal 45: 540-560 |
| 62. | Transcriptome Profiling of Citrus Fruit in Response to Huanglongbing Disease Authours: |
| | Federico Martinelli, Sandie Uratsu1, Ute Albrecht, Russell L. Reagan, Elizabeth Leicht, |
| | Raissa D'Souza, Kim D. Bowman, Abhaya M. Dandekar. |
| 63. | Wang Z, Yin Y, Hu H, Yuan Q, Peng G, Xia Y (2006) Development and application of |
| | molecular-based diagnosis for ‘Candidatus Liberibacter asiaticus’, the causal pathogen of |
| | citrus huanglongbing. Plant Pathology 55: 630-638 |
| 64. | Weckwerth W (2008) Integration of metabolomics and proteomics in molecular plant |
| | physiology--coping with the complexity by data-dimensionality reduction. Physiologia |
| | Plantarum 132: 176-189 |
| 65. | Wold S, Sjostrom M, Eriksson L (2001) PLS-regression: a basic tool of chemometrics. |
| | Chemometrics and Intelligent Laboratory Systems 58: 109-130 |
| 66. | Wolfender J-L, Ndjoko K, Hostettmann K (2003) Liquid chromatography with ultraviolet |
| | absorbance-mass spectrometric detection and with nuclear magnetic resonance spectroscopy: |
| | a powerful combination for the on-line structural investigation of plant metabolites. Journal of |
| | Chromatography. A 1000: 437-455 |
| 67. | Zhang AJ, Hartung JS (2005) Phenylacetaldehyde O-methyloxime: A volatile compound |
| | produced by grapefruit leaves infected with the citrus canker pathogen, Xanthomonas |
| | axonopodis pv. citri. Journal of Agricultural and Food Chemistry 53: 5134-5137 |
| 68. | Zhao W, Sankaran S, Ibanez AM, Dandekar AM, Davis CE (2009) Two-dimensional wavelet |
| | analysis based classification of gas chromatogram differential mobility spectrometry signals. |
| | Analytica Chimica Acta 647: 46-53 |
| 69. | Zhao, W. and C. E. Davis, Autoregressive model based feature extraction method for time |
| | shifted chromatography data. Chemometrics and Intelligent Laboratory Systems, 2009. 96(2): |
| | p. 252-257. |
| 70. | Zhao, W., J. T. Morgan, and C. E. Davis, Gas chromatography data classification based on |
| | complex coefficients of an autoregressive model. Journal of Sensors, 2008. 1(1): p. 1-8. |
| 71. | Zini CA, Augusto F, Christensen E, Caramao EB, Pawliszyn J (2002) SPME Applied to the |
| | Study of Volatile Organic Compounds Emitted by Three Species of Eucalyptus in Situ. |
| | Journal of Agricultural and Food Chemistry 50: 7199-7205 |

1. A method of diagnosing Huanglongbing disease in a citrus plant, the method comprising:
a) obtaining a sample of volatile chemical compounds released by the citrus plant;
b) measuring the quantity of one or more volatile chemical compounds, in the sample, selected from the group consisting of: linalool, tridecane (C13H28), pentanone (4-OH-4-Me-2-), hexacosane, tetradecene (1-), tricosane, geranial, tetradecanal, phenylacetaldehyde, methyl salicylate, cumacrene, caryophyllene, hexadecanol, ocimene (e-beta-), geranyl acetone, carbon dioxide, propane, 2-methyl-pentane, o-xylene, 2-ethyl-1,4-dimethyl-benzene, 1-methyl-4-(1-methylethenyl)-benzene, 2,2,3,4-tetramethyl-pentane, pentadecane (C15H32), hexenylacetate, tridecanal, vocBB 45061, vocBB 45212, 46541, 83748, 62469, 45491, 51824, 48023, 48272, 46850, 45071, 48807, 47176, 57101, 50888, 45074, and 90562; and
c) comparing the measured quantity of the one or more volatile chemical compounds to a predetermined value of the one or more volatile chemical compounds to diagnose Huanglongbing disease in the citrus plant.

2. The method of claim 1, wherein the predetermined value is determined by measuring the quantity of the one or more volatile chemical compounds released by a reference citrus plant infected with Huanglongbing disease.

3. The method of claim 1, wherein the predetermined value is determined by measuring the quantity of the one or more volatile chemical compounds released by a reference citrus plant not infected with Huanglongbing disease.

4. The method of claim 3, wherein the reference citrus plant is at the same developmental stage as the citrus plant.

5. The method of claim 1, wherein a mass spectrometer is used to measure the quantity of one or more volatile chemical compounds in the sample.

6. The method of claim 1, wherein a differential mobility spectrometer is used to measure the quantity of one or more volatile chemical compounds in the sample.

7. The method of claim 1, wherein the citrus plant is a Valencia orange plant.

8. A method of diagnosing Citrus tristeza virus (CTV) in a citrus plant, the method comprising:
a) obtaining a sample of volatile chemical compounds released by the citrus plant;
b) measuring the quantity of one or more volatile chemical compounds, in the sample, selected from the group consisting of: myrcene, carene (delta-3-), ocimene (e-beta-), hexadecanol, limonene, tetracosane, bulnesene (alpha-); and
c) comparing the measured quantity of the one or more volatile chemical compounds to a predetermined value of the one or more volatile chemical compounds to diagnose CTV disease in the citrus plant.

9. The method of claim 8, wherein the predetermined value is determined by measuring the quantity of the one or more volatile chemical compounds released by a reference citrus plant infected with CTV.

10. The method of claim 8, wherein the predetermined value is determined by measuring the quantity of the one or more volatile chemical compounds released by a reference citrus plant not infected with CTV.

11. The method of claim 10, wherein the reference citrus plant is at the same developmental stage as the citrus plant.

12. The method of claim 8, wherein a mass spectrometer is used to measure the quantity of one or more volatile chemical compounds in the sample.

13. The method of claim 8, wherein a differential mobility spectrometer is used to measure the quantity of one or more volatile chemical compounds in the sample.

14. A method of diagnosing Huanglongbing disease in a citrus plant, the method comprising:
a) obtaining a sample of nucleic acid molecules generated by the citrus plant;
b) measuring the quantity of one or more nucleic acid molecules, in the sample, selected from the group consisting of: GH3.1 (S22545043); GH3.4 (S44237769); KA02 (S44303609); salicylic acid methyl transferase (S44277040); WRKY70 (S44288591); MYB-related TF (S44256583); U-box (S22566824); HSP82 (S44237646); invertase (S35152777); terpene synthase cyclase (S22583829); NN lipid transfer protein (LTP) (S44279331); acidic cellulase 8 (S22606212); omega-6-FAD (S44244604) Acidic cellulose (S22606212); terpene synthase 1 (S44285742); ERTF2 (S44250648); 12-oxo-phytodienoate reductase (S34125138); lypoxygenase 2 (S34124539); NNLTP (NCBI accession number: EY754661.1); beta-amylase (S44303510); expansin 3 (S22533016); glucose-phosphate-transporter2 (S22591828, S22591828, S44257732, S22591828); ENT-kaurenoic acid hydroxylase 2 (S44251582); expansin3 (S22533016); alpha-amylase (S44224848); WRKY70 (S44225142, S44227900, S44288591); and
c) comparing the measured quantity of the one or more nucleic acid molecules to a predetermined value of the one or more nucleic acid molecules to diagnose Huanglongbing disease in the citrus plant.

15. The method of claim 14, wherein the nucleic acid molecules are mRNA molecules.

16. The method of claim 14, wherein the predetermined value is determined by measuring the quantity of the one or more nucleic acid molecules generated by a reference citrus plant infected with Huanglongbing disease.

17. The method of claim 14, wherein the predetermined value is determined by measuring the quantity of the one or more nucleic acid molecules generated by a reference citrus plant not infected with Huanglongbing disease.

18. The method of claim 17, wherein the reference citrus plant is at the same developmental stage as the citrus plant.
